SEQUENCE LISTING (6 sequences)

SEQ ID NO: 1 - DNA, 1997 bp, Homo sapiens
  CDS (63)..(1574)
cggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatataggaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaatcaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagcgttttgtggagtactggaccaaaaaaataaagcattataaatata

SEQ ID NO: 2 - protein, 504 aa, Homo sapiens
MAEAKTHWLGAALSLIPLIFLISGAEAASFQRNQLLQKEPDLRLENVQKFPSPEMIRALEYIENPFKRTNEIVEEQYTPQSLATLESVFQELGKLTGPNNQKRERMDEEQKLYTDDEDDIYKANNIAYEDVVGGEDWNPVEEKIESQTQEEVRDSKENIGKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 3 - DNA, 2077 bp, Homo sapiens
  CDS (63)..(356)
cggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatataggaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaatcaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagcgttttgtggagtactggaccaaaaaaataaagcattataaatata

SEQ ID NO: 4 - protein, 98 aa, Homo sapiens
  UNKNOWN
MAEAKTHWLGAALSLIPLIFLISGAEAASFQRNQLLQKEPDLRLENVQKFPSPEMIRALEYIENLRQQAHKKESLSTCNSLLCMKRIPGITPLNAQMK

SEQ ID NO: 5 - DNA, 1803 bp, Homo sapiens
  CDS (63)..(539)
cggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatataggaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagcgttttgtggagtactggaccaaaaaaataaagcattataaatata

SEQ ID NO: 6 - protein, 159 aa, Homo sapiens
  UNKNOWN
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPER

(19) United States
(12) Patent Application Publication
(10) Pub. No.: US 2005/0048504 A1
(43) Pub. Date: Mar. 3, 2005

(54) Human SGII-related gene variants associated with cancers

(76) Inventor: Ken-Shwo Dai, Hsinchu (TW)

Correspondence Address: Ladas & Parry, 26 West 61st Street, New York, NY, 10023, US

(21) Appl. No.: 10/653,685

(22) Filed: Sep. 2, 2003

(51) Int. Cl. C12Q 1/68; G01N 33/574; C07K 14/705

(52) U.S. Cl. 435/6; 435/7.23; 435/69.1; 435/320.1; 435/325; 530/350; 536/23.5

(57) ABSTRACT

The invention relates to the nucleic acid sequences of three novel human SGII-related gene variants (SGIIV1, SGIIV2 and SGIIV3) and the polypeptides encoded by SGIIV1, SGIIV2 and SGIIV3. The invention also relates to the process for producing the polypeptides encoded by SGIIV1, SGIIV2 and SGIIV3. The invention further relates to the use of the nucleic acid of SGIIV1, SGIIV2 and SGIIV3 and the polypeptide encoded by SGIIV1, SGIIV2 and SGIIV3 in diagnosing diseases associated with the deficiency of human SGIIV genes, in particular SCLC or germ cell tumors.

### FIELD OF THE INVENTION

The invention relates to the nucleic acid sequences of three novel human SGII-related gene variants (SGIIV1, SGIIV2 and SGIIV3) and the polypeptides encoded thereby, the preparation process thereof, and the uses of the same in diagnosing diseases associated with the gene variants, in particular, human cancers, e.g., small cell lung cancer or germ cell tumors.

### BACKGROUND OF THE INVENTION

Lung cancer is one of the major causes of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer, and is further divided into three main subtypes: squamous cell carcinoma, adenocarcinoma, and large cell carcinoma (Ihde and Minutesna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggests that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates. Since SCLC exhibits neuroendocrine properties, a search of the gene variants suitable for SCLC diagnosis will be focused on the genes which are associated with neuroendocrine tissue. The chromogranin-secretogranin protein family has been reported to be important for the neuroendocrine cells (Taupenot et al. (2003) N Engl J Med. 348:1134-49). Of these chromogranin-secretogranin proteins, the secretogranin II (GenBank accession # M25756; we named it SGII for the purpose of the present study) was reported to play an important role in the organization of the secretory granule matrix (Gerdes et al. (1989) J Biol Chem. 264:12009-15). This raised a possibility that the gene variants of SGII may be important targets for diagnostic markers of SCLC.

### SUMMARY OF THE INVENTION

The invention provides three SGII-related gene variants found in human SCLC, and the polypeptide sequences encoded thereby, which are useful in the diagnosis of the diseases associated with the deficiency of human SGII gene, in particular cancers, preferably SCLC or germ cell tumors.

The invention further provides expression vectors and host cells for expressing SGIIV1, SGIIV2 and SGIIV3.

The invention further provides a method for producing the polypeptides encoded by SGIIV 1, SGIIV2 and SGIIV3.

The invention further provides antibodies specifically binding to the polypeptides encoded by SGIIV1, SGIIV2 and SGIIV3.

The invention also provides methods for diagnosing the diseases associated with the deficiency of human SGII gene, in particular cancers, preferable SCLC or germ cell tumors.

### BRIEF DESCRIPTION OF THE DRAWINGS

FIG. 1A to 1E show the nucleic acid sequence of SGIIV1 (SEQ ID NO: 1) and the amino acid sequence encoded thereby (SEQ ID NO: 2).

FIG. 2A to 2E show the nucleic acid sequence of SGIIV2 (SEQ ID NO: 3) and the amino acid sequence encoded thereby (SEQ ID NO: 4).

FIG. 3A to 3D show the nucleic acid sequence of SGIIV3 (SEQ ID NO: 5) and the amino acid sequence encoded thereby (SEQ ID NO: 6).

FIG. 4A to 4T show the nucleotide sequence alignment between human SGII gene and SGIIV1, SGIIV2 and SGIIV3.

FIG. 5A to 5F show the amino acid sequence alignment among human SGII and the polypeptides encoded by SGIIV1, SGIIV2 and SGIIV3.

### DETAILED DESCRIPTION OF THE INVENTION

According to the invention, all technical and scientific terms used have the same meanings as commonly understood by persons skilled in the art.

The term “antibody,” as used herein, denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, an antibody consists of four subunits including two heavy chains and two light chains. The internal surface shape and charge distribution of the antibody binding domain are complementary to the features of an antigen. Thus, an antibody can specifically act against the antigen in an immune response.

The term “base pair (bp),” as used herein, denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402),” as used herein, denotes programs for evaluation of homologies between a query sequence (amino or nucleic acid) and a test sequence as described by Altschul et al. (Nucleic Acids Res. 25: 3389-3402, 1997). Specific BLAST programs are described as follows:
(1) BLASTN compares a nucleotide query sequence against a nucleotide sequence database;
(2) BLASTP compares an amino acid query sequence against a protein sequence database;
(3) BLASTX compares the six-frame conceptual translation products of a query nucleotide sequence against a protein sequence database;
(4) TBLASTN compares a query protein sequence against a nucleotide sequence database translated in all six reading frames; and
(5) TBLASTX compares the six-frame translations of a nucleotide query sequence against the six-frame translations of a nucleotide sequence database.

The term “cDNA,” as used herein, denotes nucleic acids that are synthesized from a mRNA template using reverse transcriptase.

The term “cDNA library,” as used herein, denotes a library composed of complementary DNAs which are reverse-transcribed from mRNAs.

The term “complement,” as used herein, denotes a polynucleotide sequence capable of forming base pairing with another polynucleotide sequence. For example, the sequence 5′-ATGGACTTACT-3′ binds to the complementary sequence 5′- AGTAAGTCCAT-3′.

The term “deletion,” as used herein, denotes a removal of a portion of one or more amino acid residues/nucleotides from a gene.

The term “expressed sequence tags (ESTs),” as used herein, denotes short (200 to 500 base pairs) nucleotide sequence that derives from either 5′ or 3′ end of a cDNA.

The term “expression vector,” as used herein, denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell,” as used herein, denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition,” as used herein, denotes the addition of a portion of one or more amino acid residues/nucleotides to a gene.

The term “in silico,” as used herein, denotes a process of using computational methods (e.g., BLAST) to analyze DNA sequences.

The term “polymerase chain reaction (PCR),” as used herein, denotes a method which increases the copy number of a nucleic acid sequence using a DNA polymerase and a set of primers (about 20-30 bp oligonucleotides complementary to each strand of DNA) under suitable conditions (successive rounds of primer annealing, strand elongation, and dissociation).

The term “primer,” as used herein, denotes a single-stranded synthetic oligonucleotide designed to hybridize to a particular template DNA sequence. The forward primer is the one complementary to one strand at the 5′-end of the DNA sequence. The reverse primer is the one complementary to the other strand at the 3′-end of the DNA sequence.

The term “protein” or “polypeptide,” as used herein, denotes a sequence of amino acids in a specific order that can be encoded by a gene or by a recombinant DNA. It can also be chemically synthesized.

The term “nucleic acid sequence” or “polynucleotide,” as used herein, denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR),” as used herein, denotes a process which transcribes mRNA to complementary DNA strand using reverse transcriptase followed by polymerase chain reaction to amplify the specific fragment of DNA sequences.

The term “transformation,” as used herein, denotes a process describing the uptake, incorporation, and expression of exogenous DNA by prokaryotic host cells.

The term “transfection,” as used herein, a process describing the uptake, incorporation, and expression of exogenous DNA by eukaryotic host cells.

The term “variant,” as used herein, denotes a fragment of sequence (nucleotide or amino acid) inserted or deleted by one or more nucleotides/amino acids.

In the first aspect, the subject invention provides the nucleotide sequences of SGIIV1, SGIIV2 and SGIIV3, and the polypeptides encoded by the three novel human SGII-related gene variants and fragments thereof.

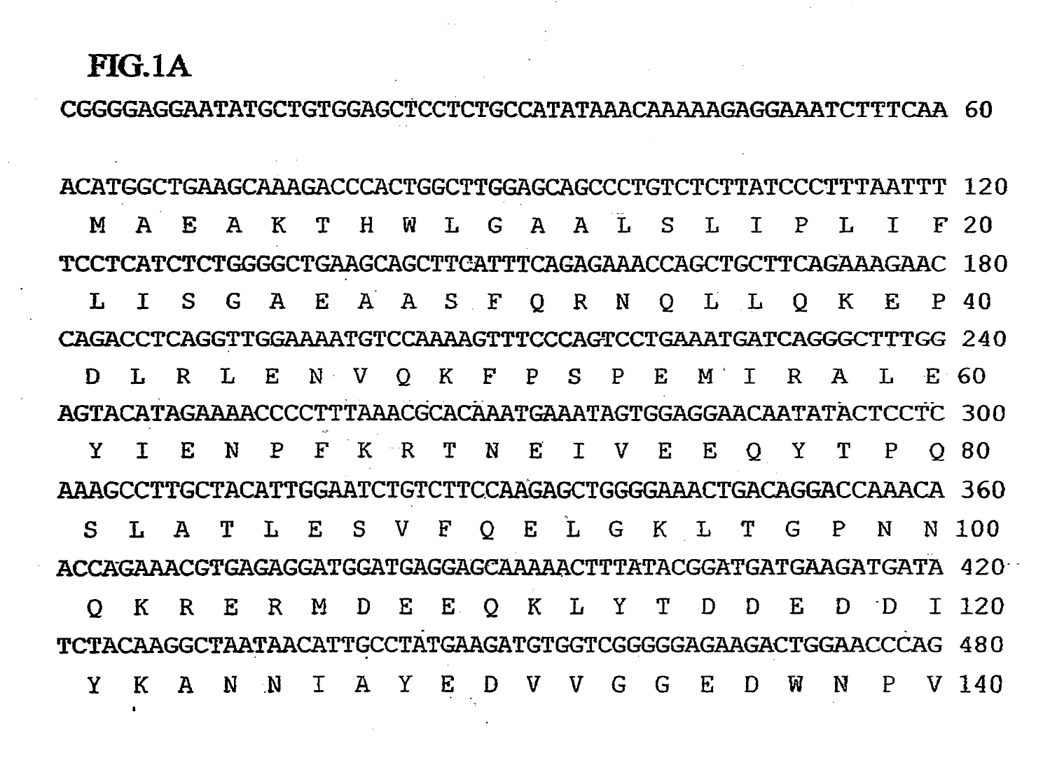

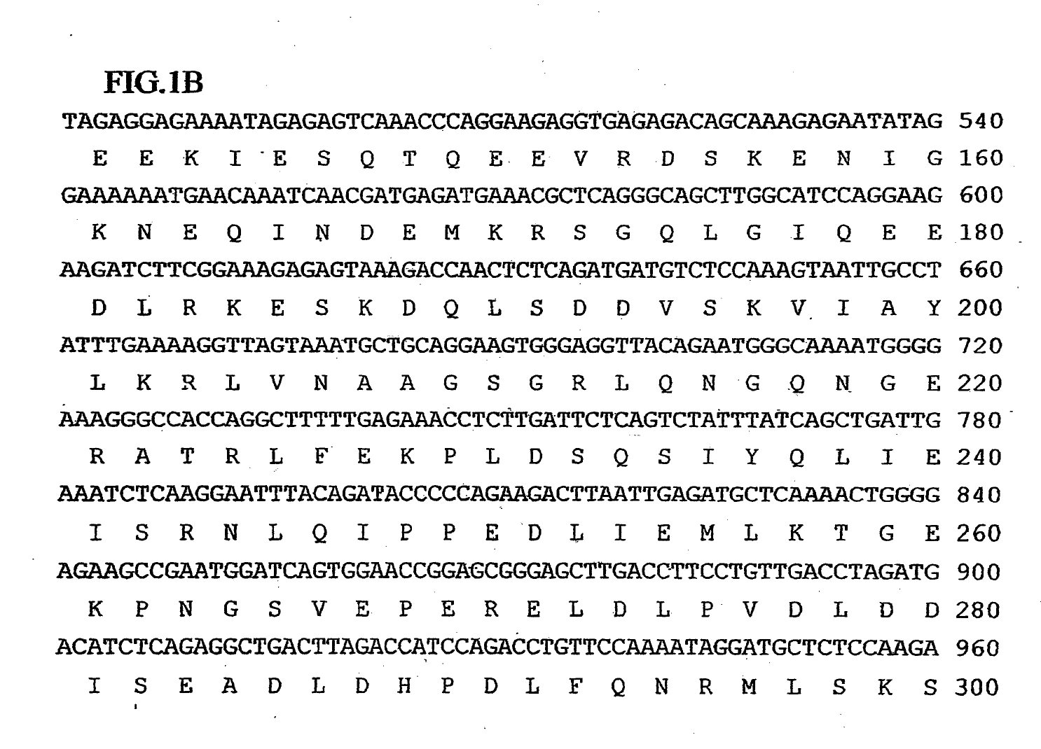

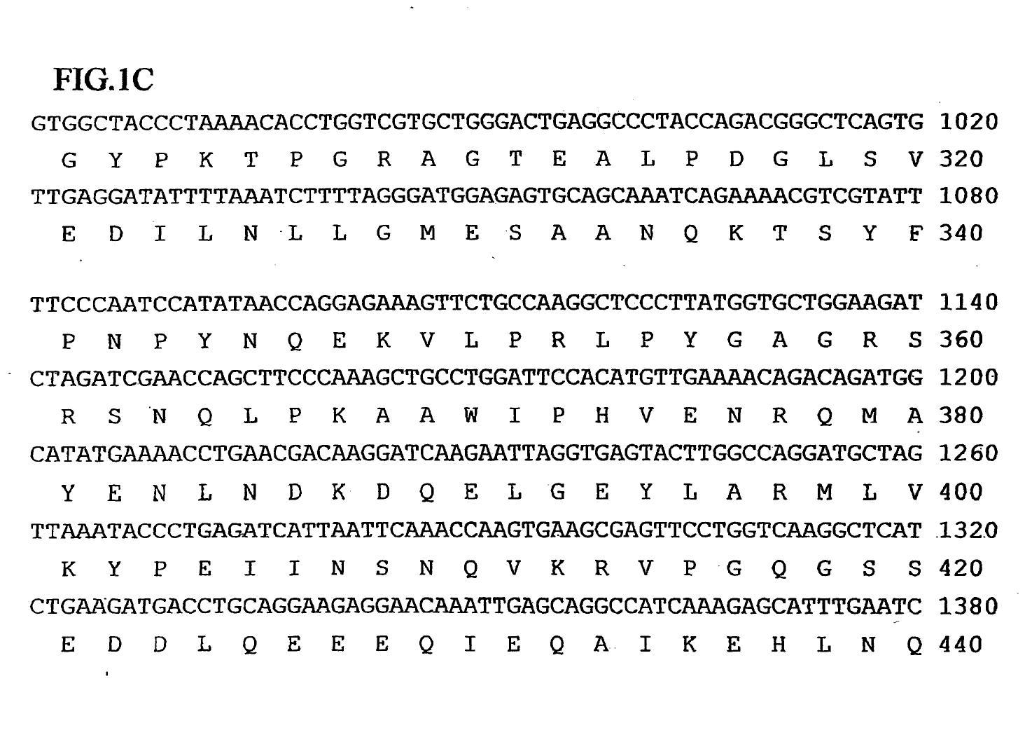

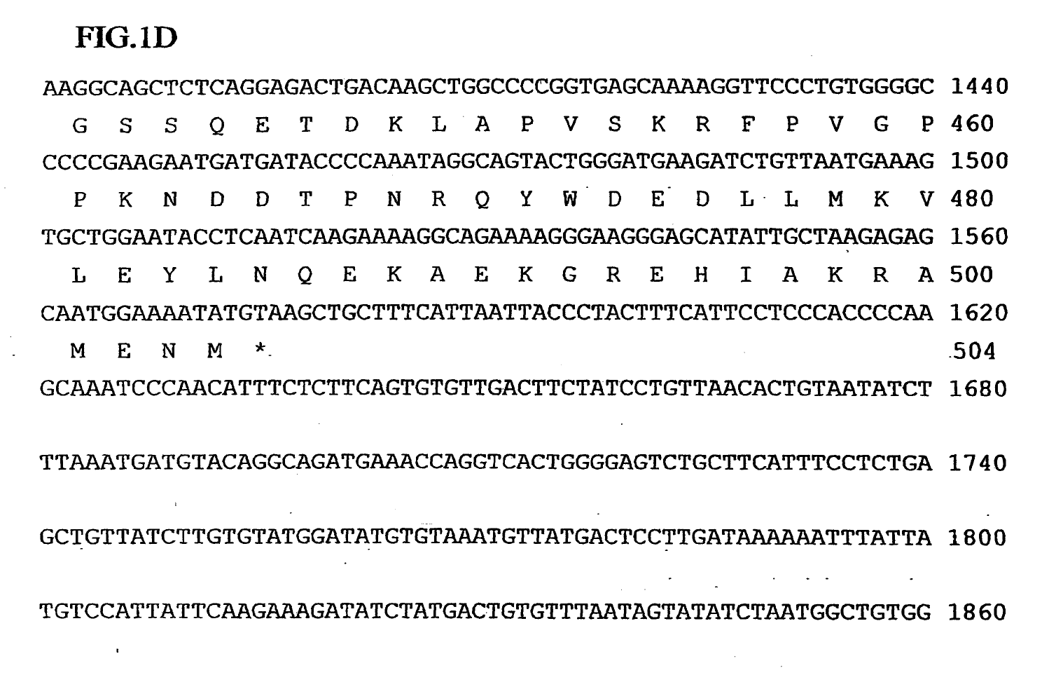

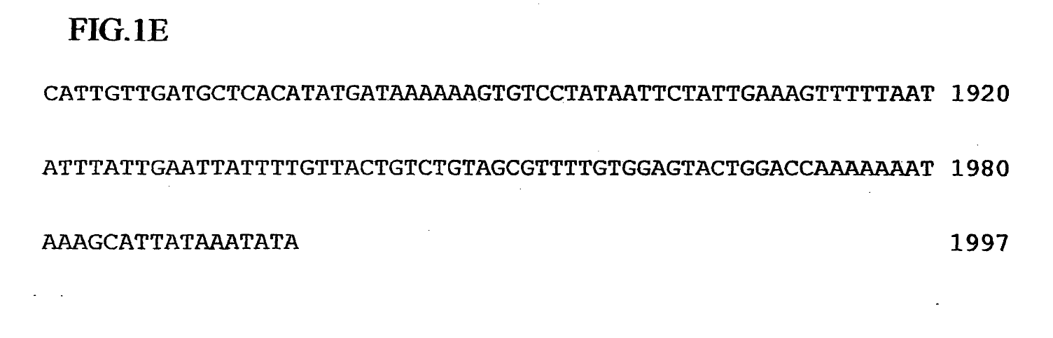

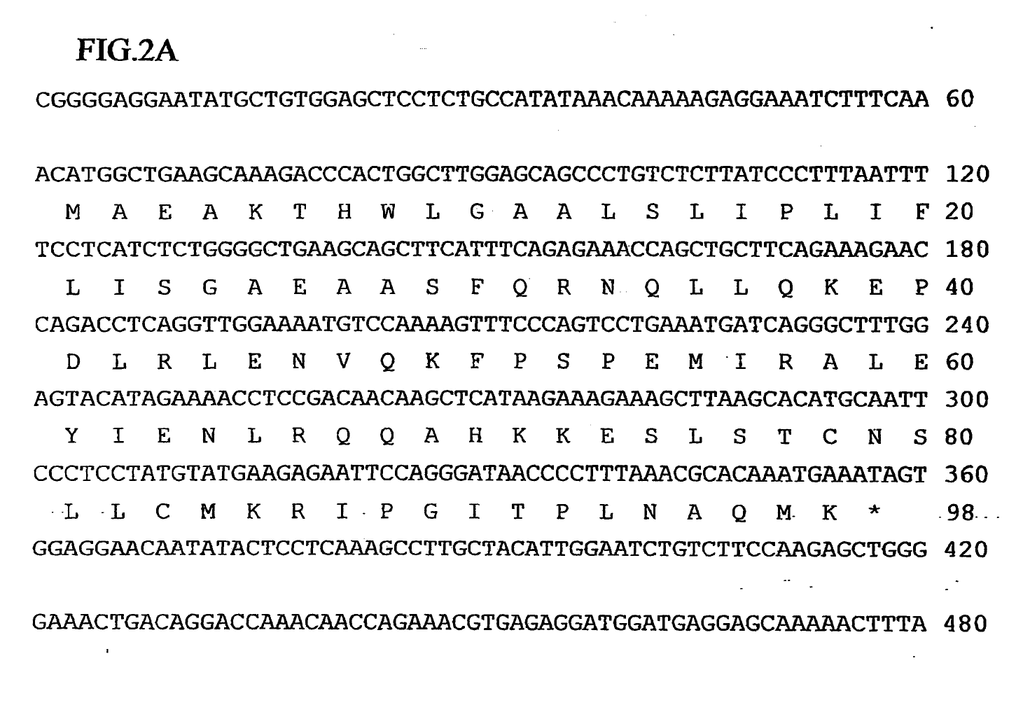

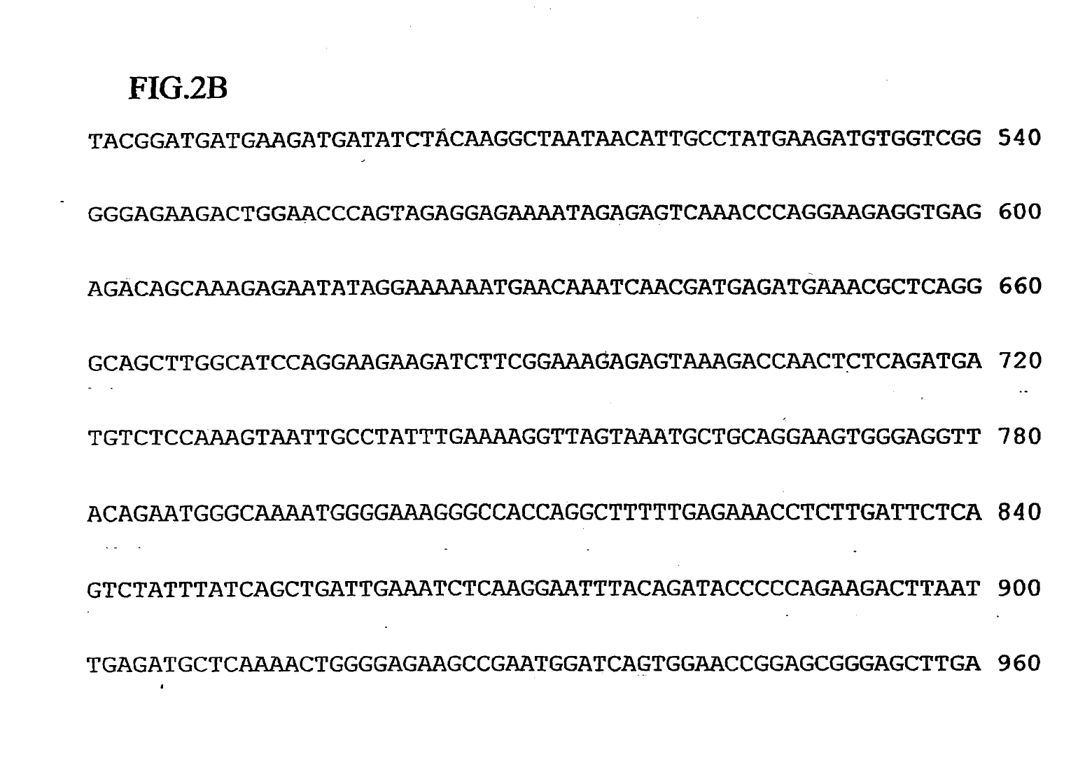

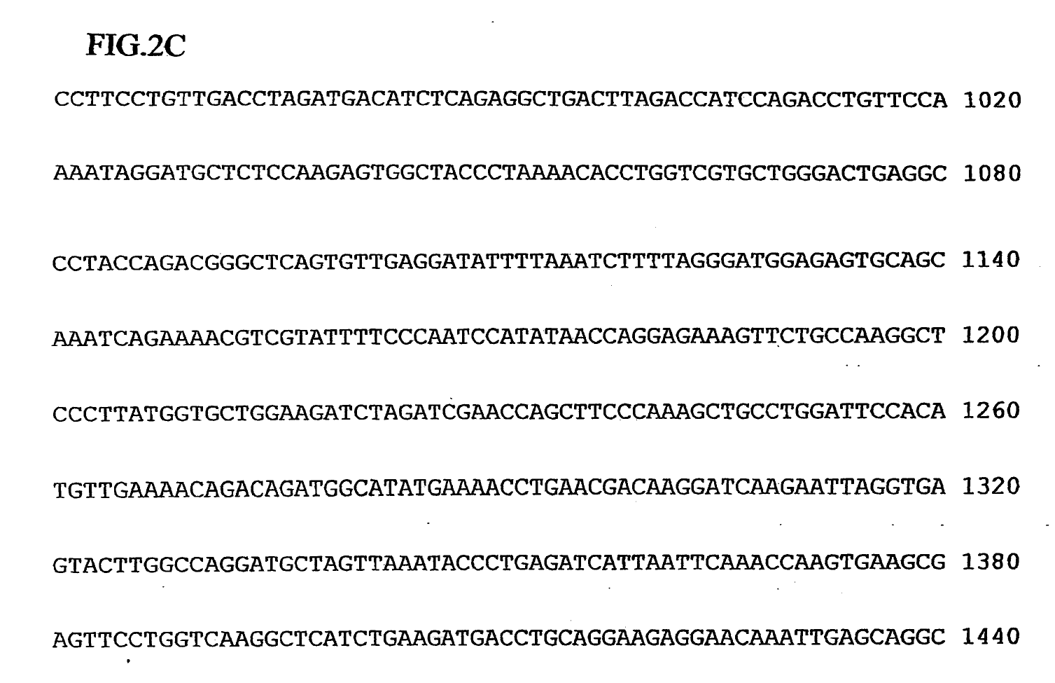

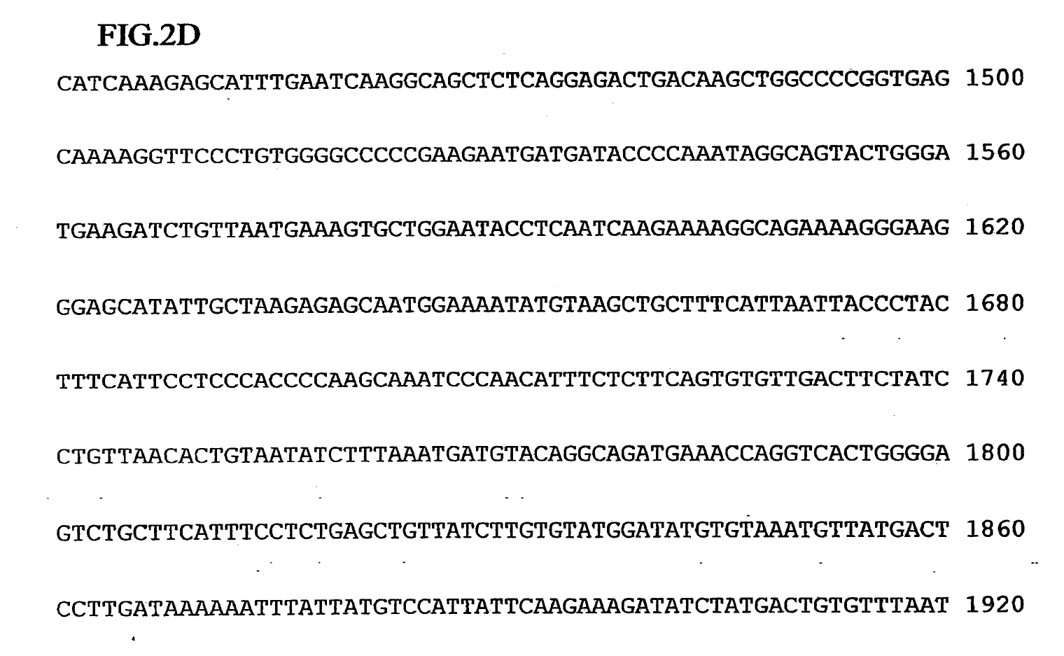

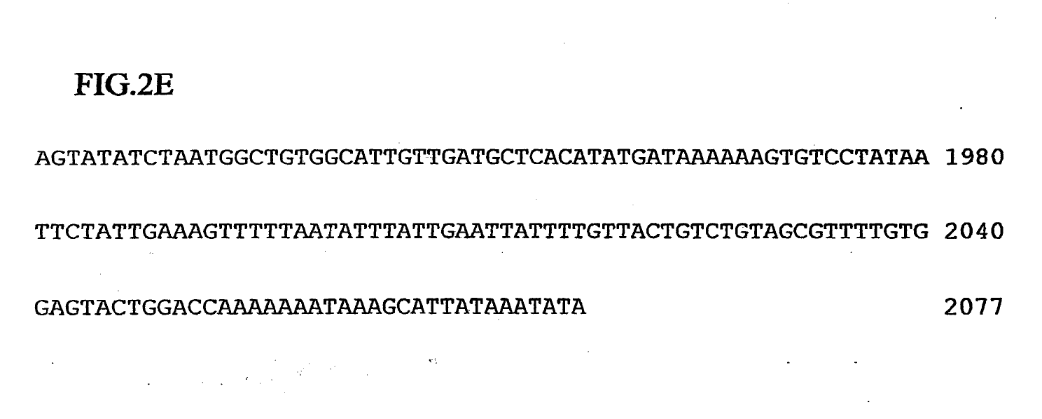

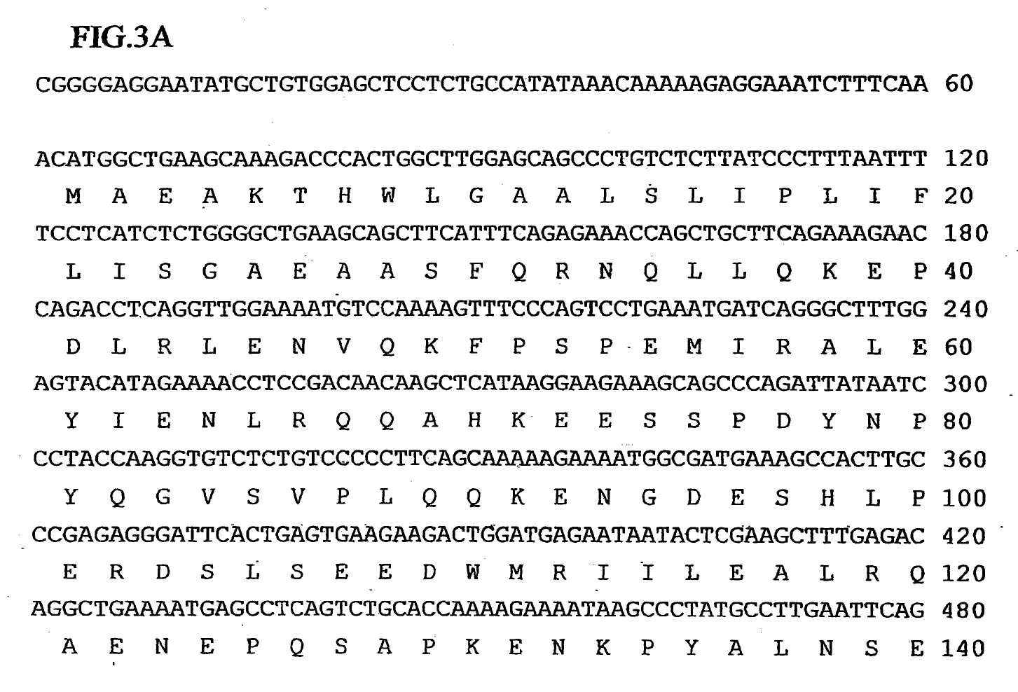

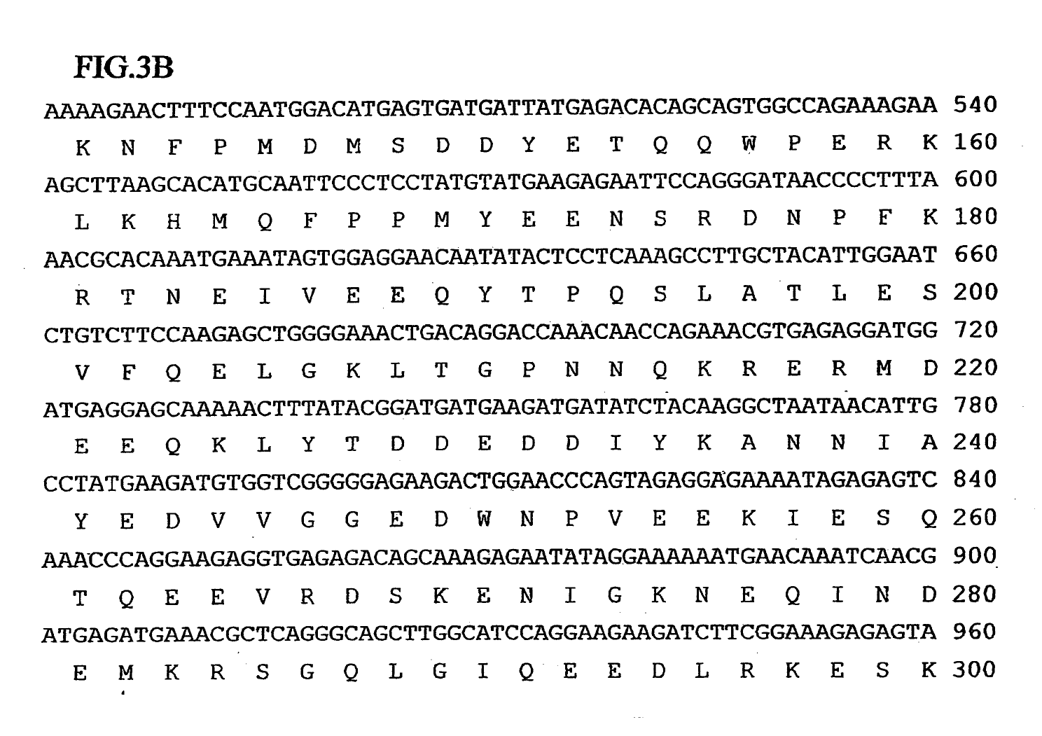

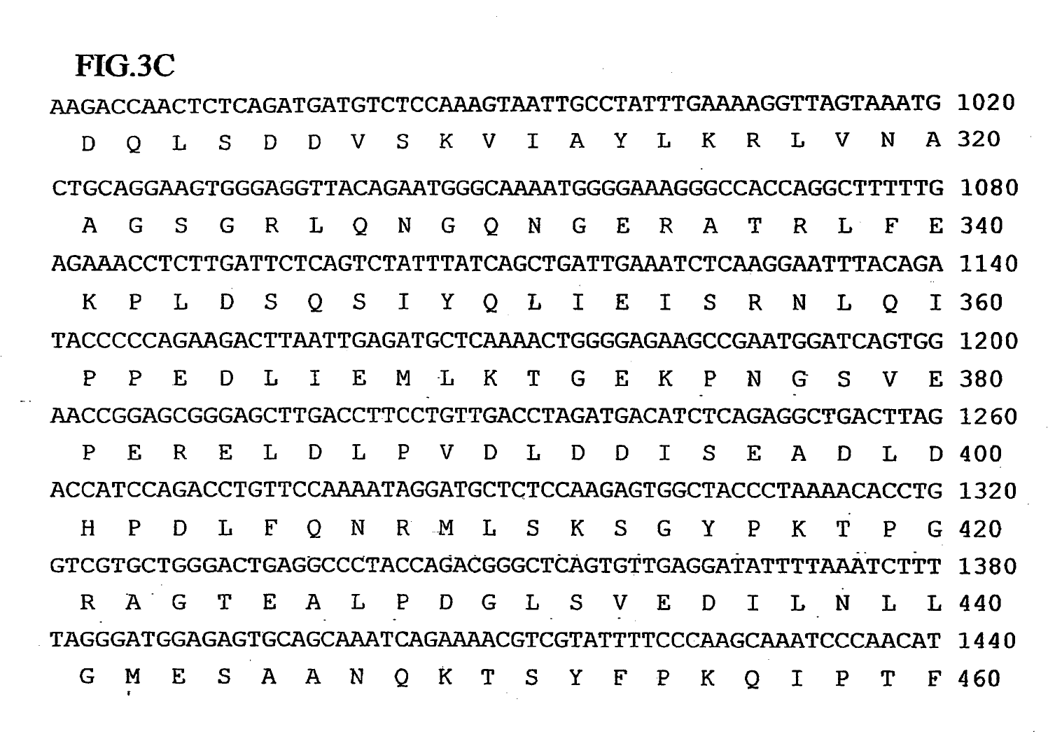

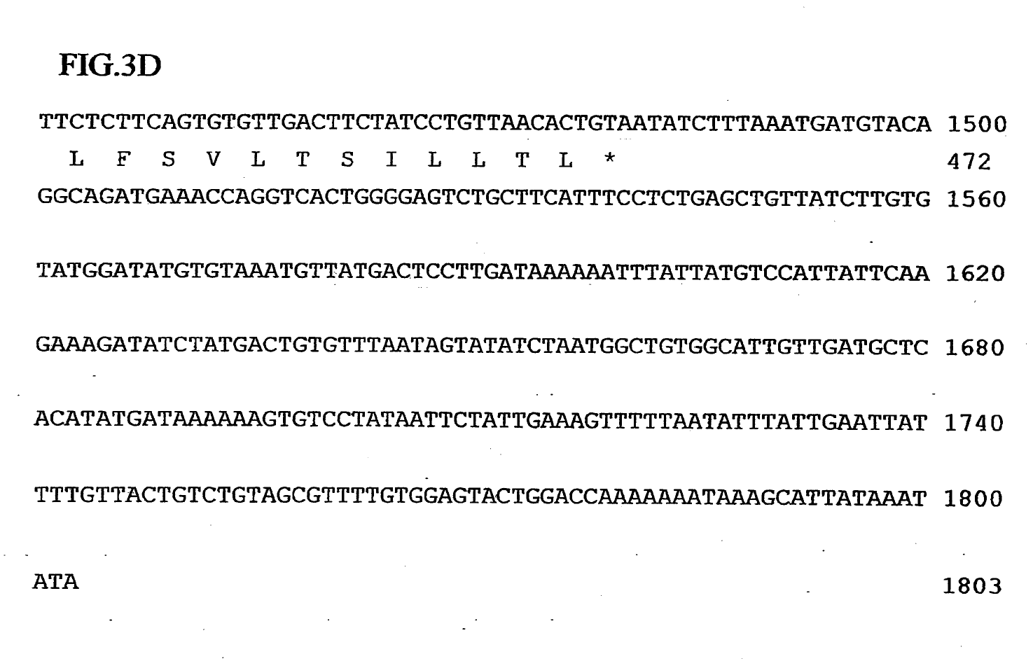

According to the invention, human SGII cDNA sequence was used to query a human SCLC EST database using BLAST program to search for SGII-related gene variants. Three human cDNA partial sequences (i.e., ESTs) deposited in the databases showing similarity to SGII were isolated and sequenced. These clones (named SGIIV1, SGIIV2 and SGIIV3) were isolated. FIGS. 1, 2 and 3 show the nucleic acid sequences (SEQ ID NOs: 1, 3 and 5) of the variants (SGIIV1, SGIIV2 and SGIIV3) and the corresponding amino acid sequences (SEQ ID NOs: 2, 4 and 6) encoded thereby.

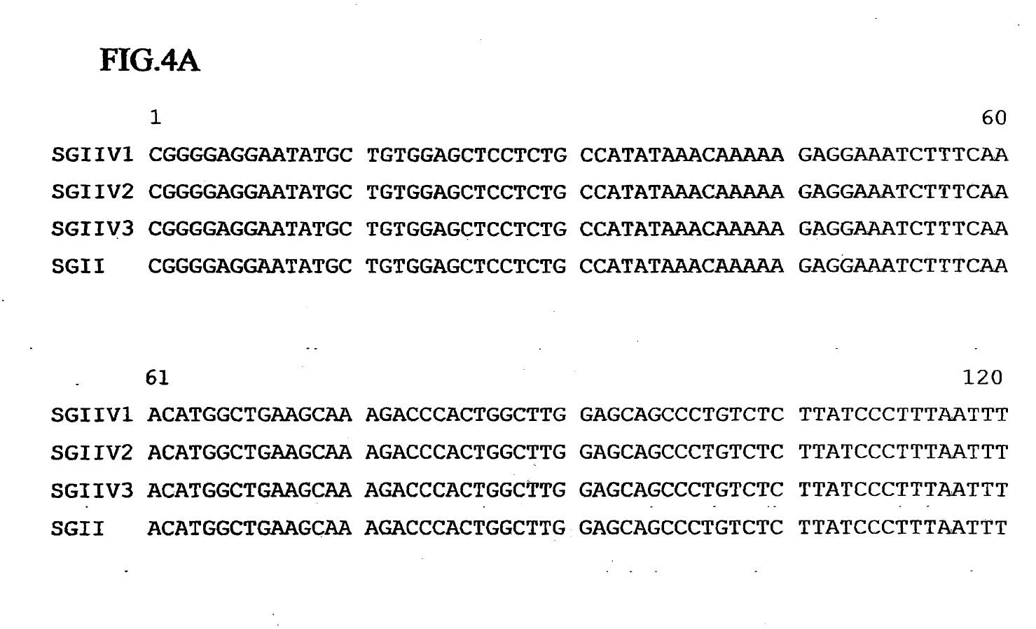

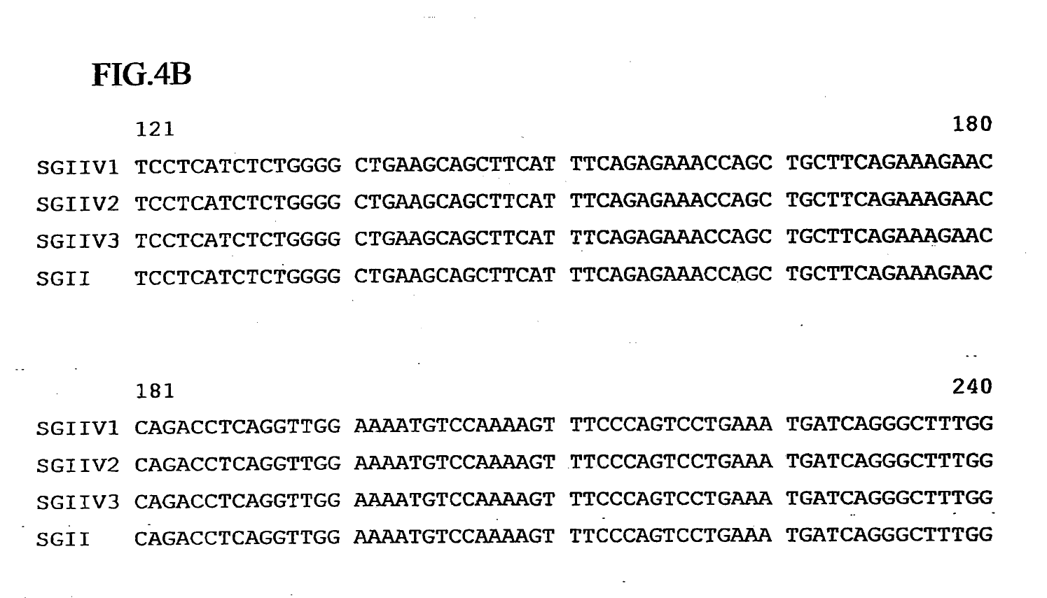

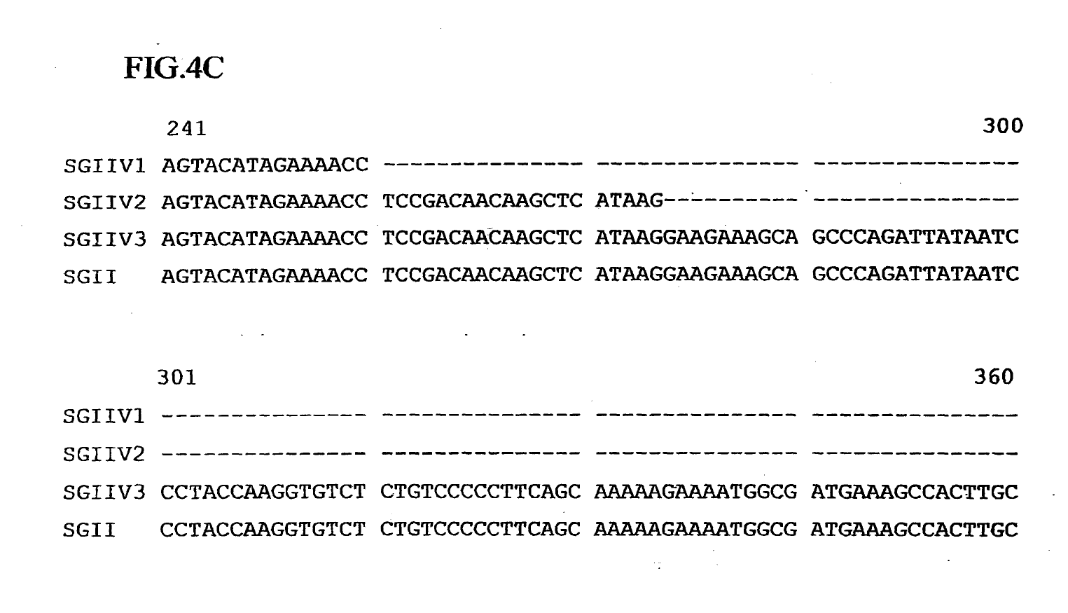

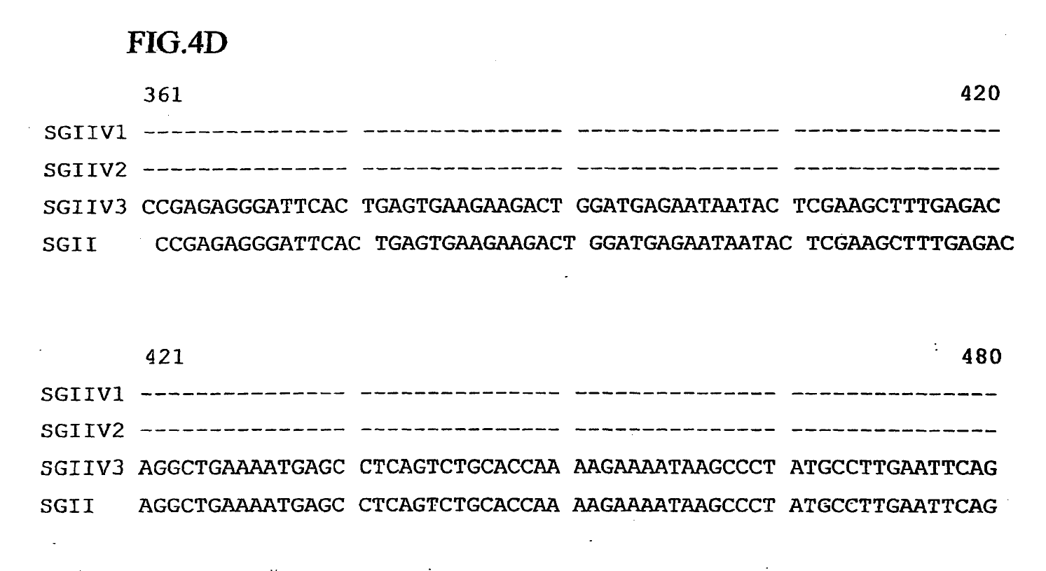

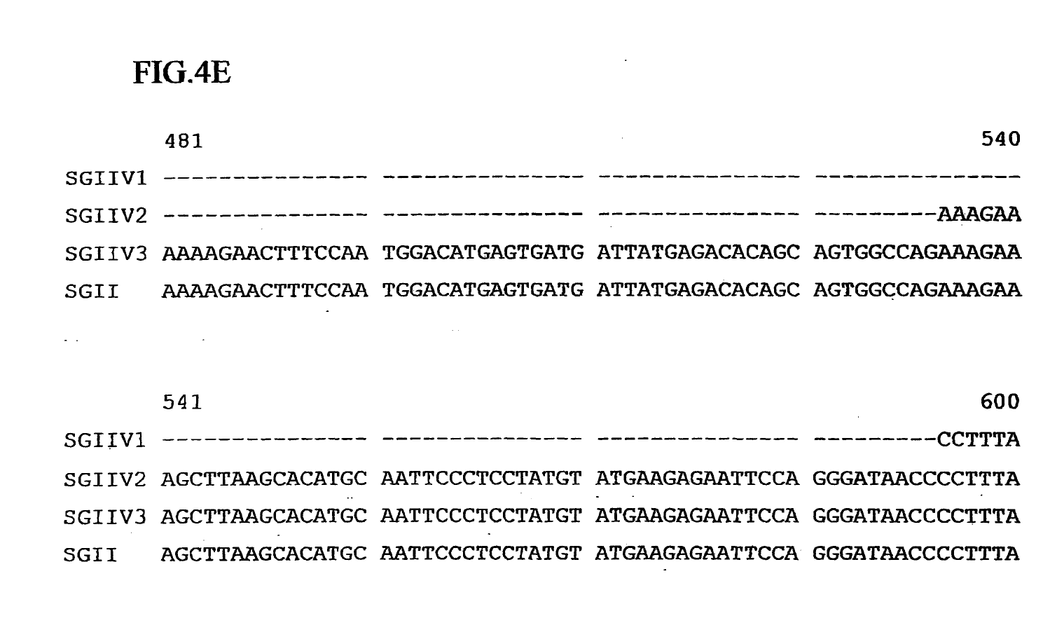

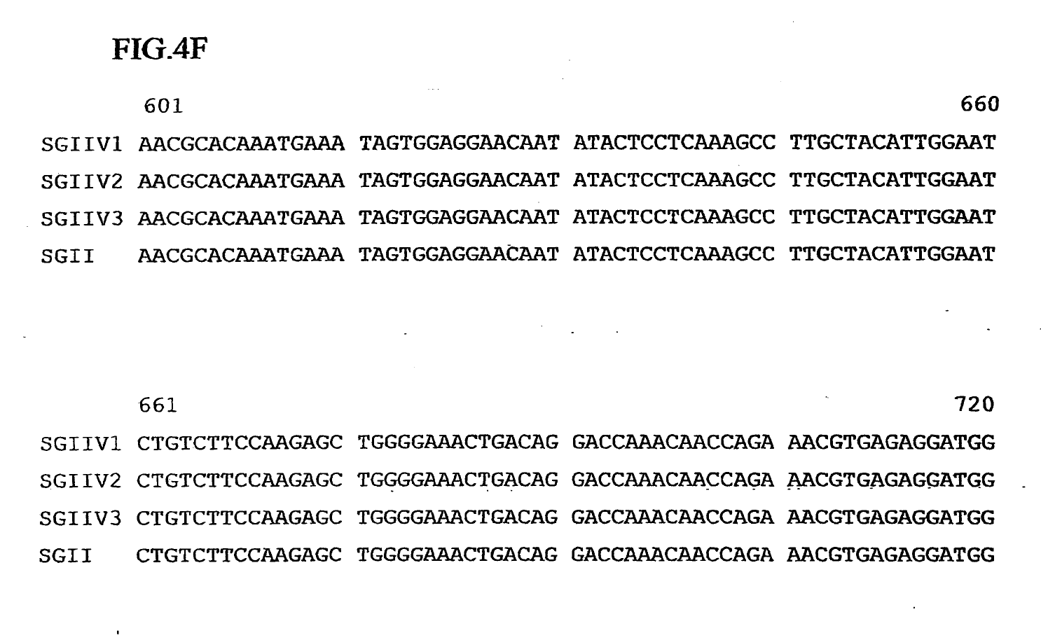

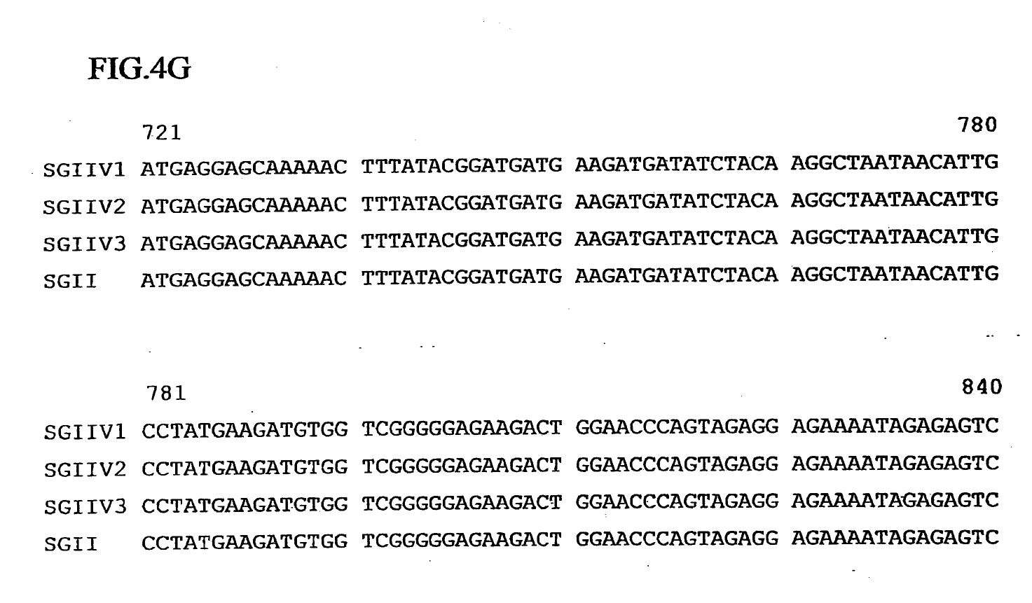

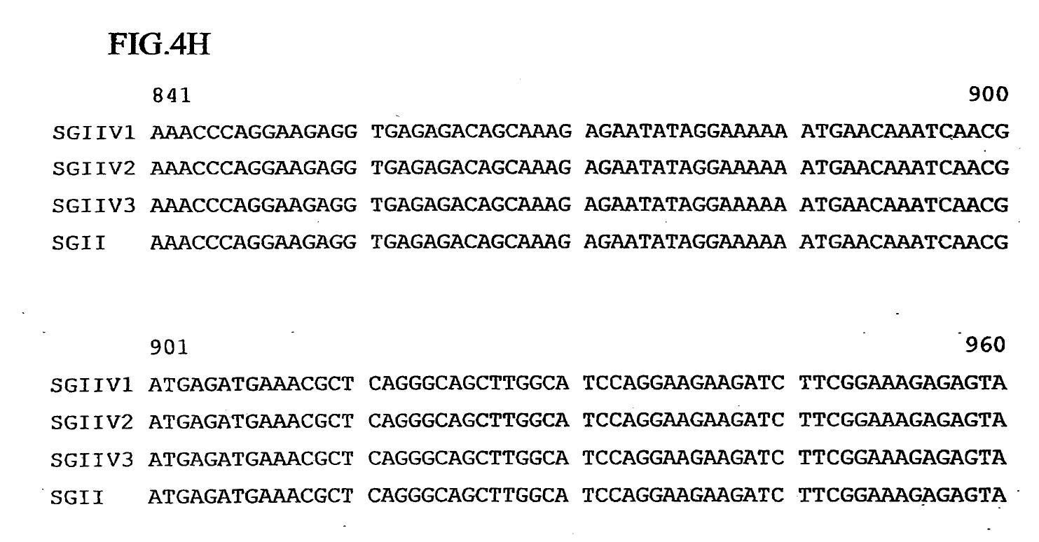

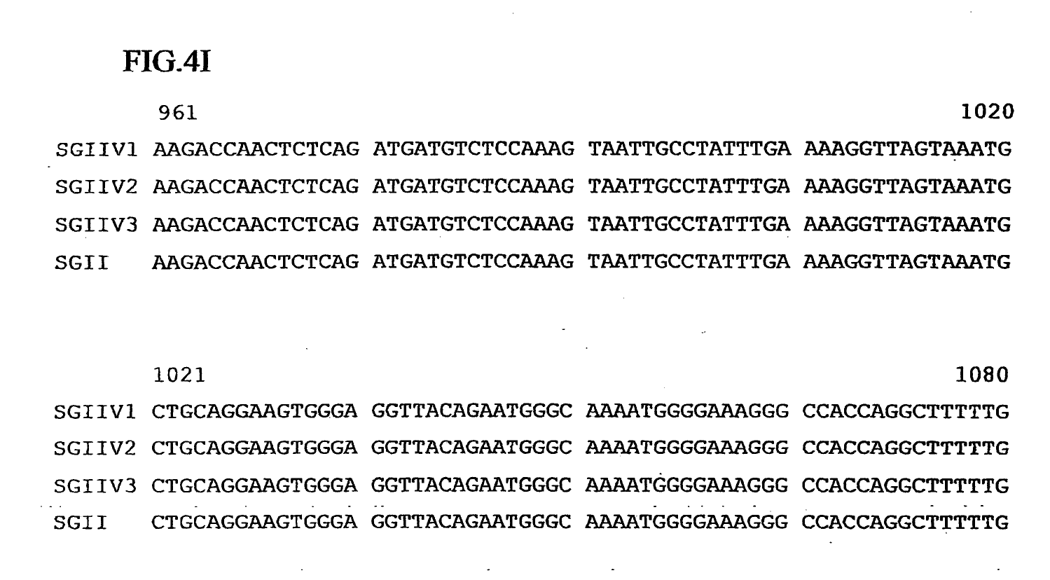

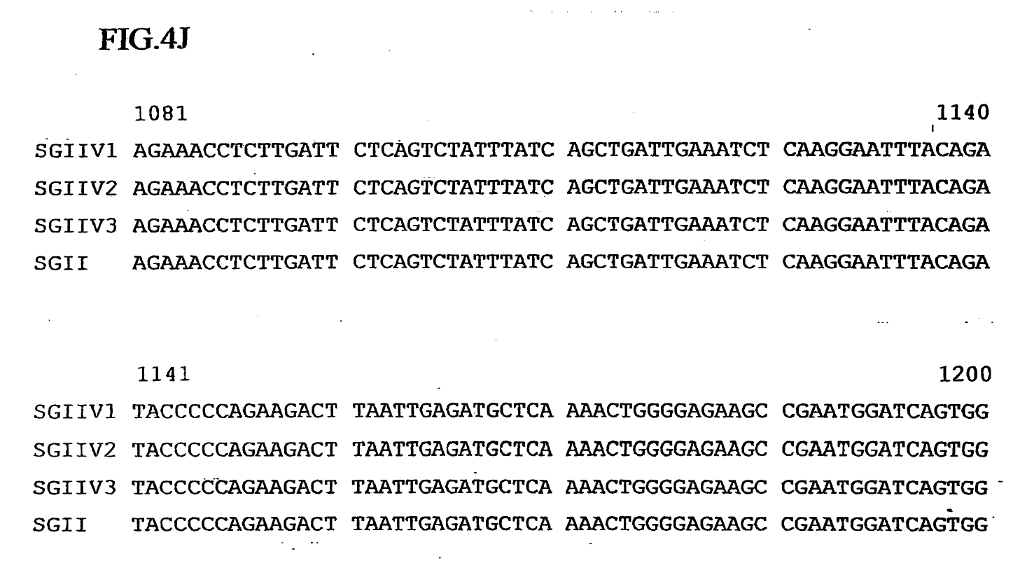

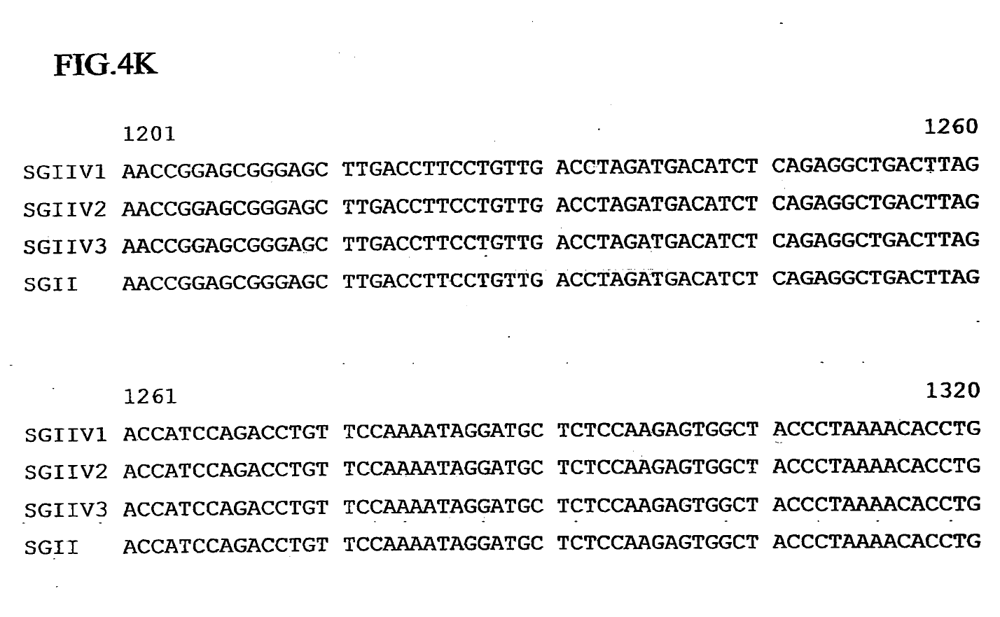

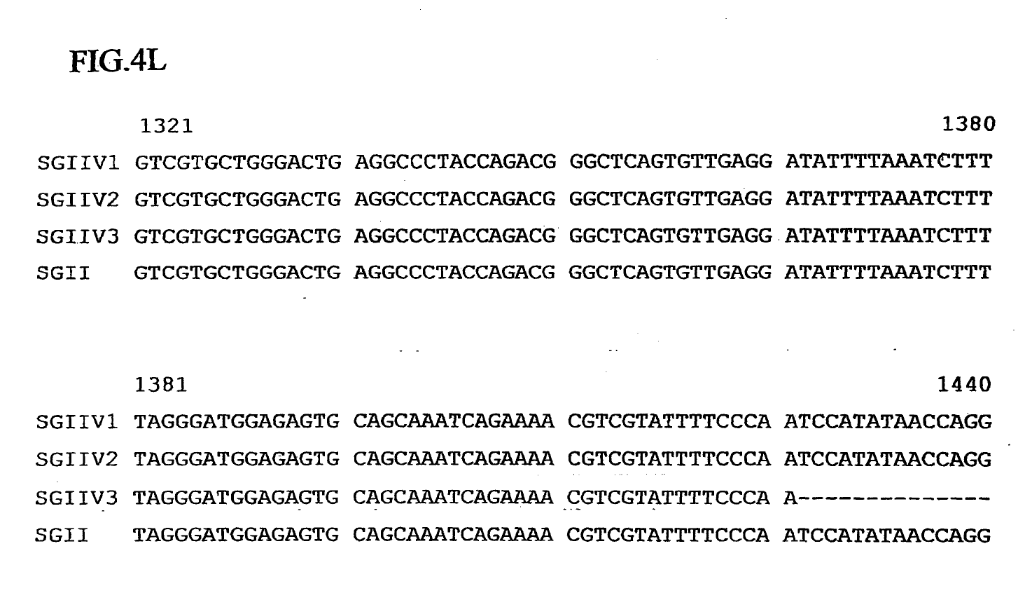

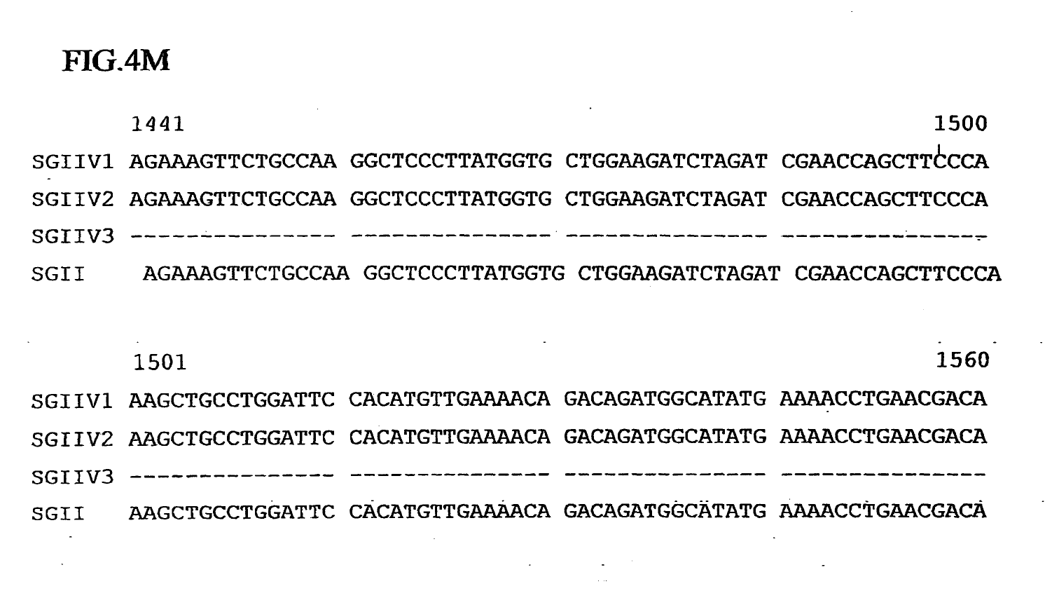

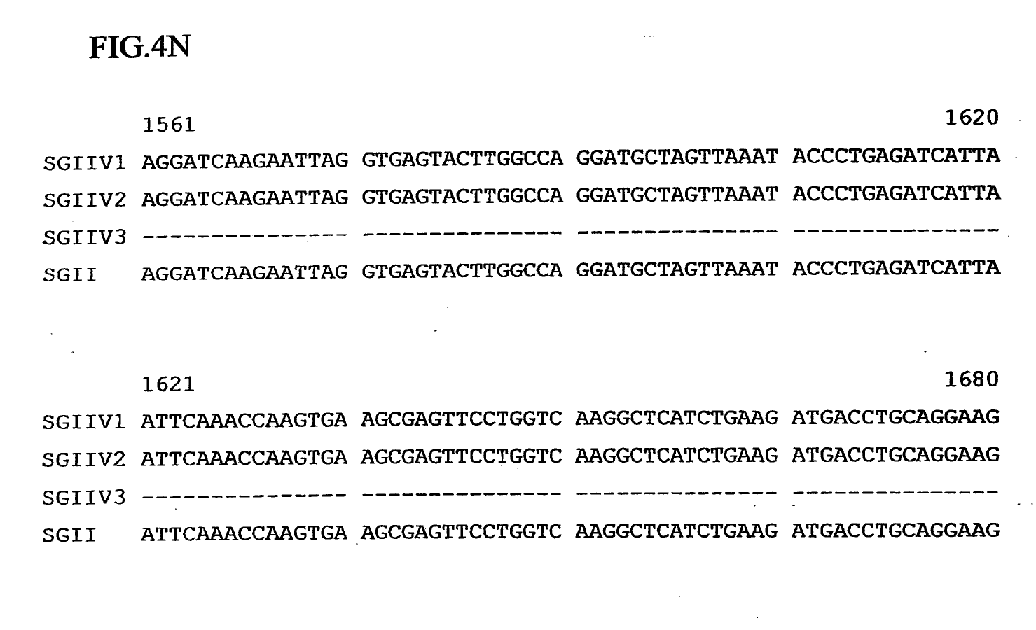

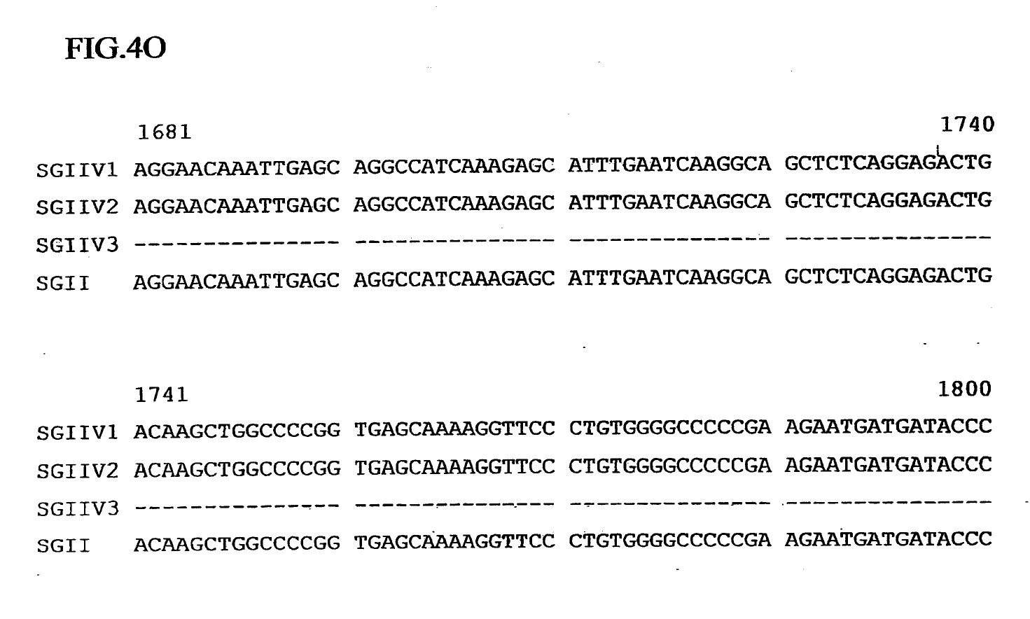

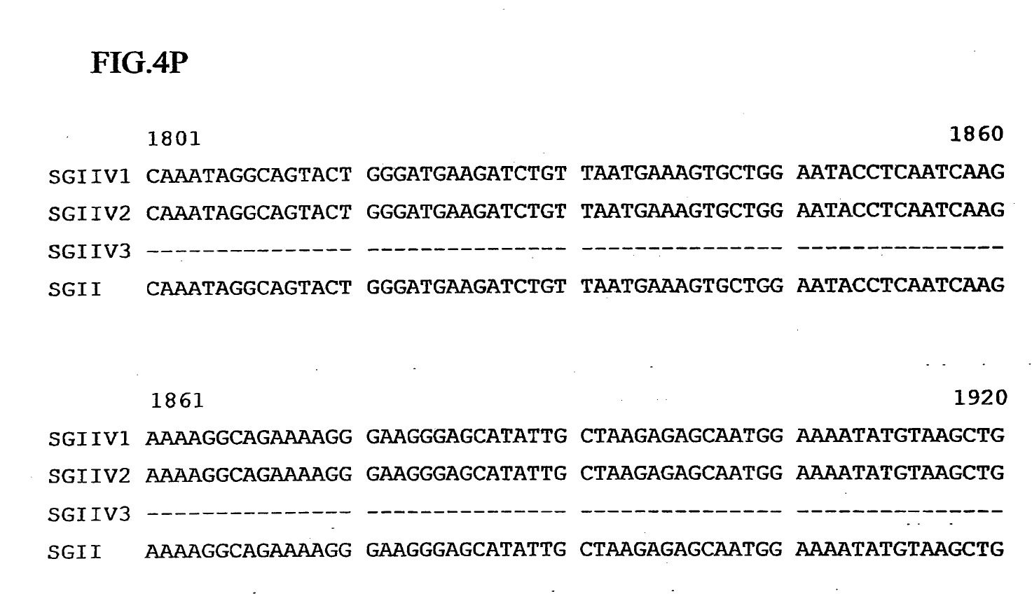

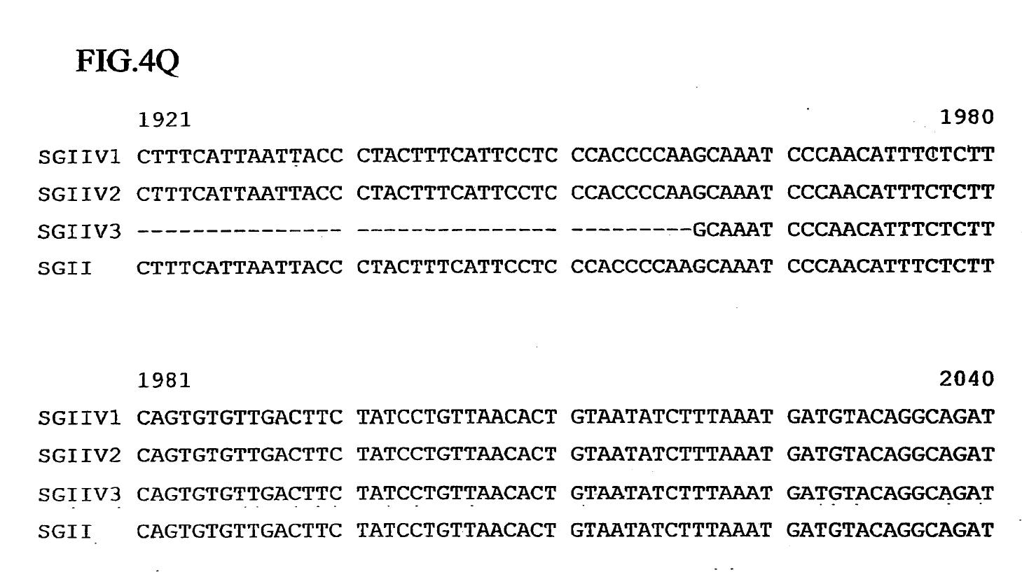

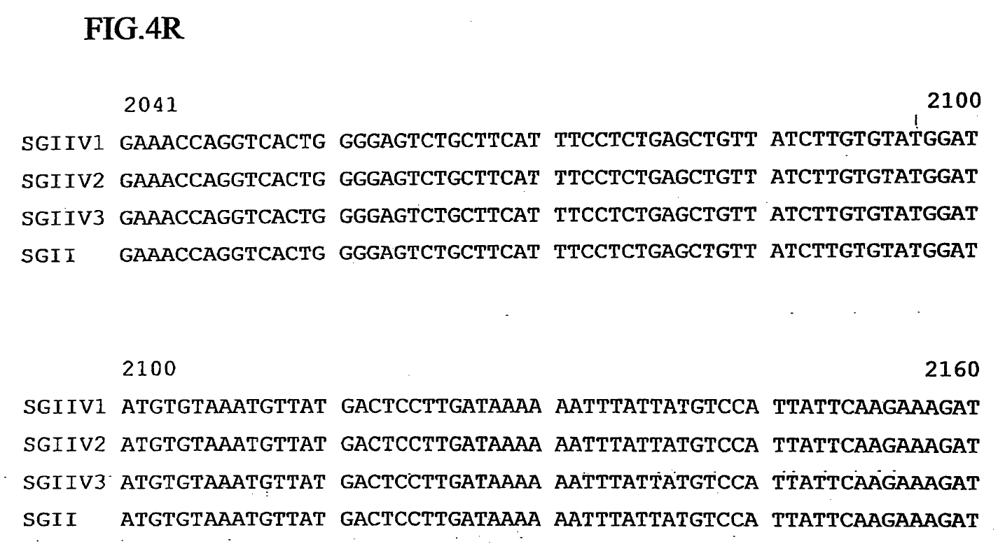

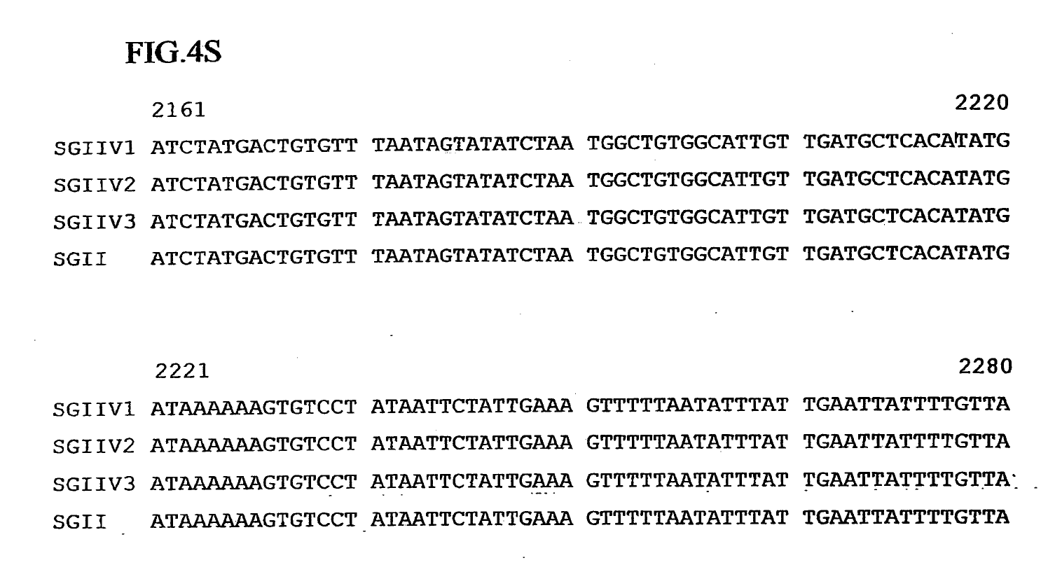

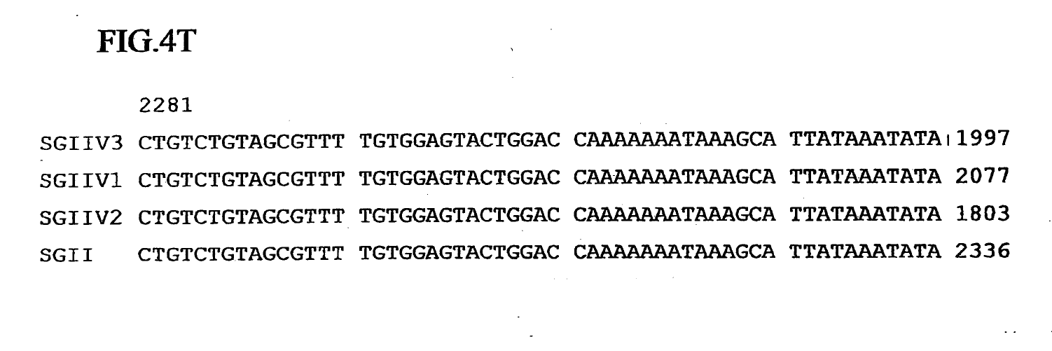

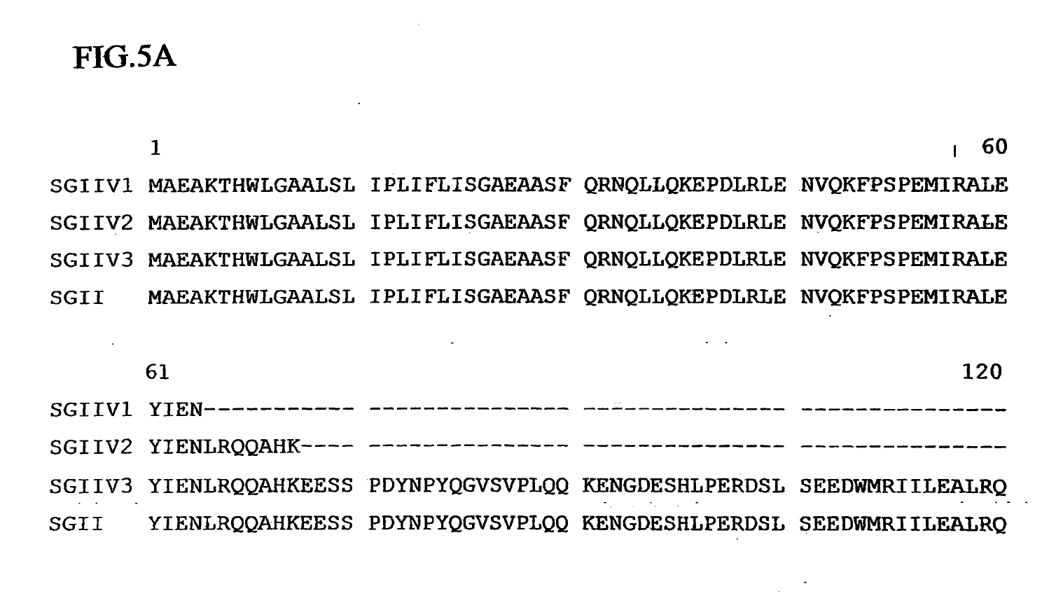

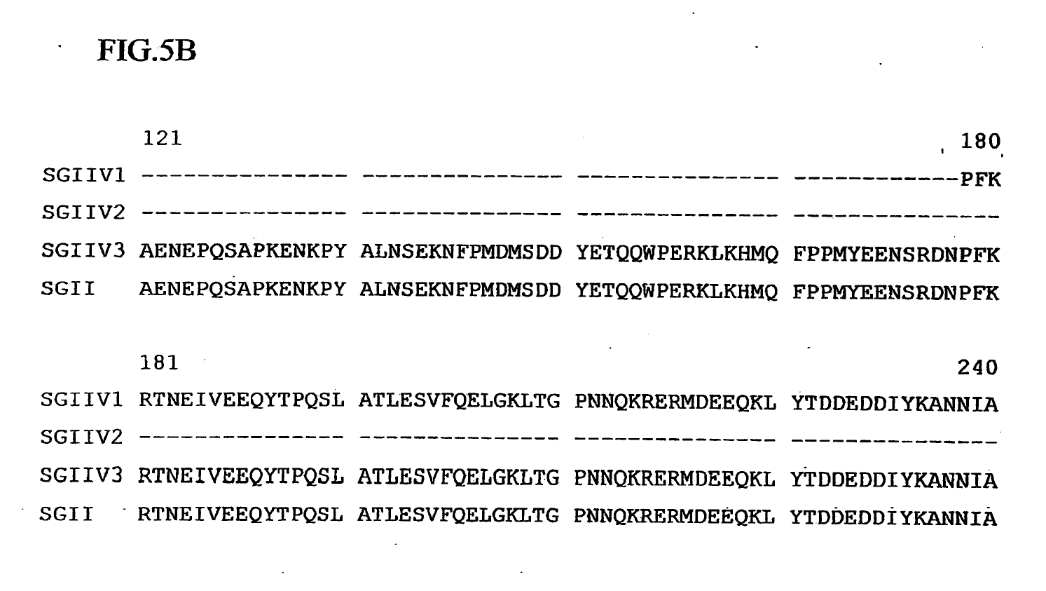

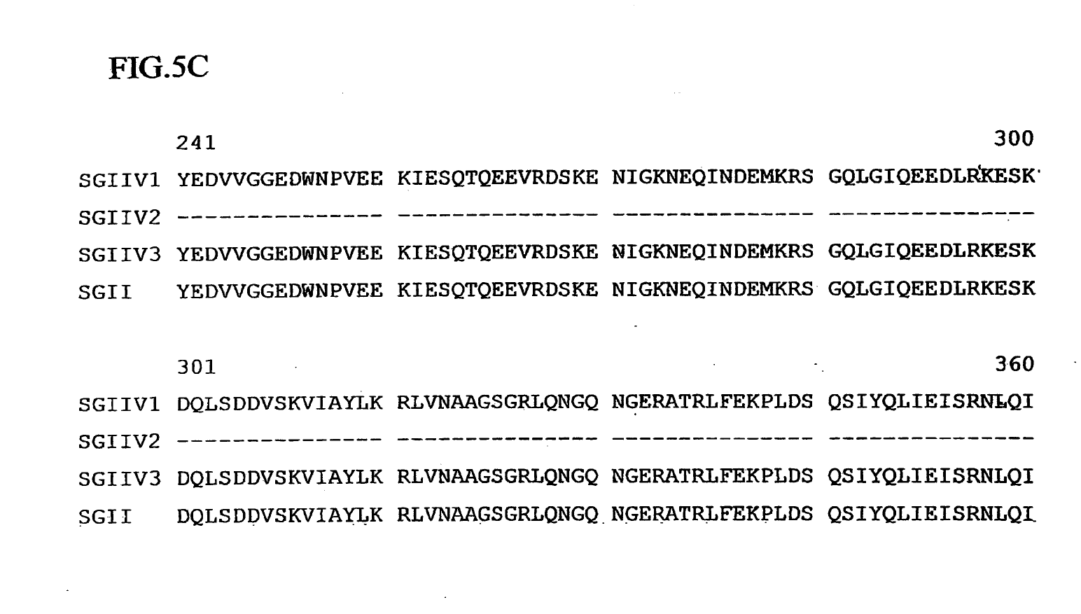

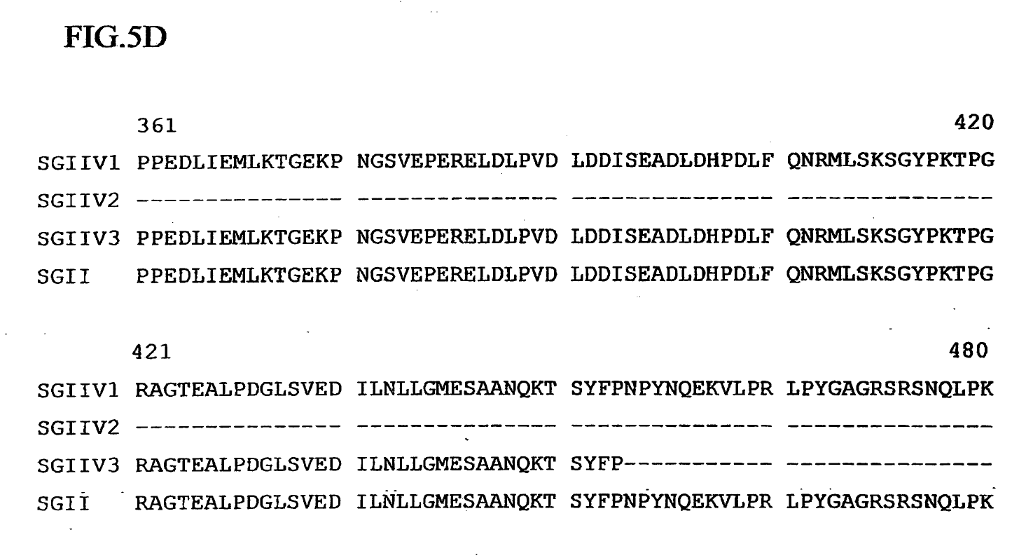

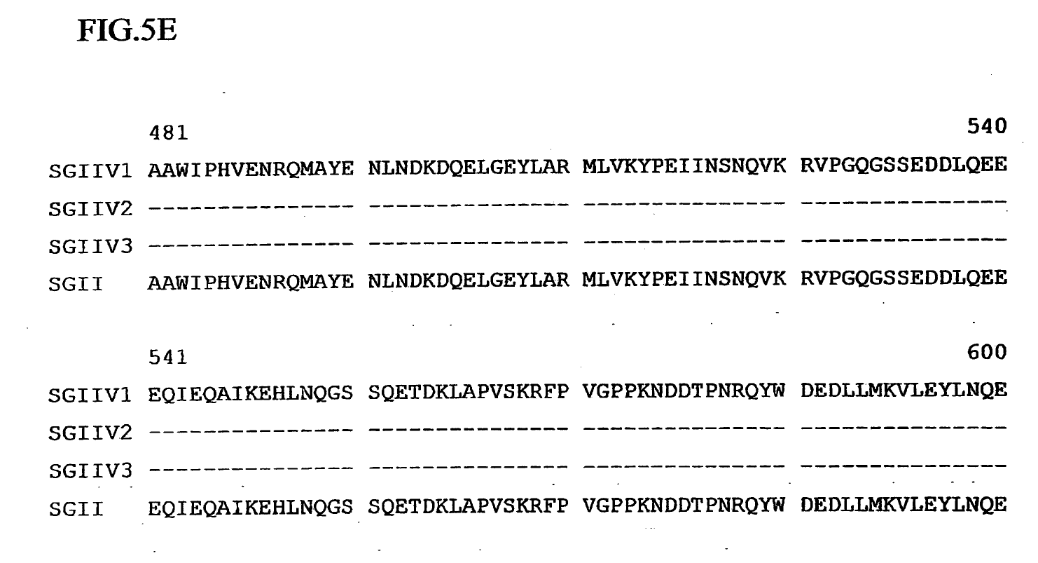

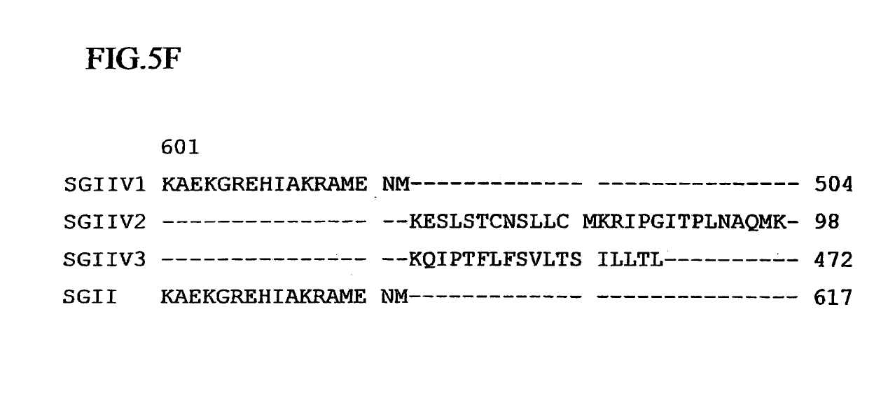

The full-length of the SGIIV1 cDNA is a 1997 bp clone containing a 1512 bp open reading frame (ORF) extending from nucleotides 63 p to 1574, which corresponds to an encoded protein of 504 amino acid residues with a predicted molecular mass of 57.5 kDa. The full-length of the SGIIV2 cDNA is a 2077 bp clone containing a 294 bp ORF extending from nucleotides 63 to 356, which corresponds to an encoded protein of 98 amino acid residues with a predicted molecular mass of 11.1 kDa. The full-length of the SGIIV3 cDNA is a 1803 bp clone containing a 1416 bp ORF extending from nucleotides 63 to 1478, which corresponds to an encoded protein of 472 amino acid residues with a predicted molecular mass of 54.0 kDa. To determine the variations (insertion/deletion) in sequences of SGIIV1, SGIIV2 and SGIIV3 cDNA clones, an alignment of SGII nucleotide/amino acid sequence with these clones was performed (FIGS. 4 and 5). The results indicate that three genetic deletions were found in the aligned sequences. This information demonstrates that SGIIV1 is a 339 bp deletion in the sequence of SGII from nucleotides 256-594; SGIIV2 is a 259 bp deletion in the sequence of SGII from nucleotides 276-534; and SGIIV3 is a 533 bp deletion in the sequence of SGII from nucleotides 1427-1959.

In the invention, a search of ESTs deposited in dbEST (Boguski et al., (1993) Nat Genet. 4: 332-3) at NCBI was performed. Three ESTs were found to confirm the missing region described in SGIIV1, SGIIV2 and SGIIV3. One EST (GenBank accession number A1655028), which confirmed the absence of a 339 bp region in SGIIV1 nucleotide sequences, was found to have been isolated from a pooled germ cell tumors cDNA library. This suggests that the absence of the 339 bp nucleotide fragment located between nucleotides 255-256 of SGIIV1 may be a useful marker for SCLC or germ cell tumors diagnosis. One EST (GenBank accession number AI671205), which confirmed the absence of a 259 bp region in SGIIV2 nucleotide sequences, was found to have been isolated from a pooled germ cell tumors cDNA library. This suggests that the absence of the 259 bp nucleotide fragment located between nucleotides 275-276 of SGIIV2 may be a useful marker for SCLC or germ cell tumors diagnosis. One EST (GenBank accession number AA936920), which confirmed the absence of a 533 bp region in SGIIV3 nucleotide sequences, was found to have been isolated from a pooled germ cell tumors cDNA library. This suggests that the absence of 533 bp nucleotide fragment located between nucleotides 1426-1427 of SGIIV3 is an important marker in association with SCLC or germ cell tumors.

Therefore, the nucleotide fragments comprising nucleotides 253-258, preferably nucleotides 240-269 of SGIIV1, nucleotides 273-278, preferably nucleotides 261-290 of SGIIV2 or nucleotides 1424-1429, preferably nucleotides 1413-1442 of SGIIV3 may be used as probes for determining the presence of the variants under highly stringent conditions. An alternative approach is that any set of primers for amplifying the fragment containing nucleotides 253-258, preferably nucleotides 240-269 of SGIIV1, nucleotides 273-278, preferably nucleotides 261-290 of SGIIV2 or nucleotides 1424-1429, preferably nucleotides 1413-1442 of SGIIV3 may be used for determining the presence of the variants.

According to the present invention, the polypeptides encoded by human SGII-related gene variants (SGIIV1, SGIIV2 and SGIIV3) and fragments thereof may be produced through genetic engineering techniques. In this case, they are produced by appropriate host cells that have been transformed by DNAs that code the polypeptides or fragments thereof. The nucleotide sequence encoding the polypeptide of the human SGII-related gene variants or fragment thereof is inserted into an appropriate expression vector, i.e., a vector which contains the necessary elements for the transcription and translation of the inserted coding sequence in a suitable host. The nucleic acid sequence is inserted into the vector in a manner that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing the sequences encoding the polypeptides of the human SGII-related gene variants and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express the polypeptide-coding sequence. These include, but are not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and most preferably, the bacterium is E. coli.

Alternatively, the polypeptides encoded by human SGII-related gene variants or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 43 1A peptide synthesizer (Perkin-Elmer).

According to the present invention, the fragments of the polypeptides and nucleic acid sequences of the human SGII-related gene variants are used as immunogens and primers or probes, respectively. It is preferable to use the purified fragments of the human SGII-related gene variants. The fragments may be produced by enzyme digestion, chemical cleavage of isolated or purified polypeptide or nucleic acid sequences, or chemical synthesis and then may be, isolated or purified. Such isolated or purified fragments of the polypeptides and nucleic acid sequences can be used directly as immunogens and primers or probes, respectively.

The present invention further provides the antibodies which specifically bind one or more out-surface epitopes of the polypeptides encoded by human SGII-related gene variants.

According to the present invention, immunization of mammals with immunogens described herin, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. No. 4,271,145 and U.S. Pat. No. 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for the production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci U S A 93: 12394-9; Liu et al., (1997) Nat Genet 16:328-9; Siffert et al., (1998) Nat Genet 18: 45 to 8; Lukas et al., (2001) Cancer Res 61: 3212 to 9). Based on the cDNA libraries of the matched ESTs, SGIIV1, SGIIV2 and SGIIV3 can be specifically associated with SCLC or germ cell tumors. Thus, the expression level of SGIIV1, SGIIV2 and SGIIV3, each relative to SGII, may be a useful indicator for screening of patients suspected of having cancers, or more specifically, the SCLC or germ cell tumors. This suggests that the index of relative expression level (mRNA or protein) may be associated with an increased susceptibility to cancers, more preferably, SCLC or germ cell tumors. Fragments of SGIIV1, SGIIV2 and SGIIV3 transcripts (mRNAs) may be detected by RT-PCR approach. Polypeptides encoded by the SGII-related gene variants may be determined by the binding of antibodies to these polypeptides. These approaches may be performed in accordance with conventional methods well known by persons skilled in the art.

The subject invention also provides methods for diagnosing the diseases associated with the deficiency of human SGII gene in a mammal, in particular, lung cancer, e.g., SCLC and germ cell tumors.

The method for diagnosing the diseases associated with the deficiency of human SGII genes may be performed by detecting the nucleotide sequences of SGIIV1, SGIIV2 or SGIIV3 of the invention, which comprises the steps of: (1) extracting total RNA of cells obtained from a mammal; (2) amplifying the RNA by reverse transcriptase-polymerase chain reaction (RT-PCR) with a set of primers to obtain a cDNA comprising the fragments comprising nucleotides 253-258, preferably nucleotides 240-269 of SEQ ID NO: 1 or nucleotides 273-278, preferably nucleotides 261-290 of SEQ ID NO: 3 or nucleotides 1424-1429, preferably nucleotides 1413-1442 of SEQ ID NO: 5; and (3) detecting whether the cDNA sample is obtained. If necessary, the amount of the obtained cDNA sample may be detected.

In this embodiment, a forward primer may be designed to have a sequence comprising nucleotides 253-258, preferably nucleotides 240-269 of SEQ ID NO: 1 and a reverse primer may be designed to have a sequence complementary to the nucleotides of SEQ ID NO: 1 at any other locations downstream of nucleotide 258, preferably nucleotide 269; or a forward primer has a sequence comprising nucleotides 273-278, preferably nucleotides 261-290 of SEQ ID NO: 3 and a reverse primer has a sequence complementary to the nucleotides of SEQ ID NO: 3 at any other locations downstream of nucleotide 278, preferably nucleotide 290; or a forward primer has a sequence comprising nucleotides 1424-1429, preferably nucleotides 1413-1442 of SEQ ID NO: 5 and a reverse primer has a sequence complementary to the nucleotides of SEQ ID NO: 5 at any other locations downstream of nucleotide 1429, preferably nucleotide 1442. Alternatively, the reverse primer may be designed to have a sequence complementary to the nucleotides of SEQ ID NO: 1 containing nucleotides 253-258, preferably nucleotides 240-269 and the forward primer may be designed to have a sequence comprising the nucleotides of SEQ ID NO: 1 at any other locations upstream of nucleotide 253, preferably nucleotide 240; or the reverse primer has a sequence complementary to the nucleotides of SEQ ID NO: 3 containing nucleotides 273-278, preferably nucleotides 261-290 and the forward primer has a sequence comprising the nucleotides of SEQ ID NO: 3 at any other locations upstream of nucleotide 273, preferably nucleotide 261; or the reverse primer has a sequence complementary to the nucleotides of SEQ ID NO: 5 containing nucleotides 1424-1429, preferably nucleotides 1413-1442 and the forward primer has a sequence comprising the nucleotides of SEQ ID NO: 5 at any other locations upstream of nucleotide 1424, preferably nucleotide 1413. In this case, only SGIIV1, SGIIV2 and SGIIV3 will be amplified.

Alternatively, the forward primer may be designed to have a sequence comprising the nucleotides of SEQ ID NO: 1 at any locations upstream of nucleotide 253 and the reverse primer may be designed to have a sequence complementary to the nucleotides of SEQ ID NO: 1 at any other locations downstream of nucleotide 258; or the forward primer has a sequence comprising the nucleotides of SEQ ID NO: 3 at any locations upstream of nucleotide 273 and the reverse primer has a sequence complementary to the nucleotides of SEQ ID NO: 3 at any other locations downstream of nucleotide 278; or the forward primer has a sequence comprising the nucleotides of SEQ ID NO: 5 at any locations upstream of nucleotide 1424 and the reverse primer has a sequence complementary to the nucleotides of SEQ ID NO: 5 at any other locations downstream of nucleotide 1429. In this case, SGIIV1, SGIIV2 or SGIIV3 together with SGII in a sample will be amplified. The length of the PCR fragment from SGIIV1 will be 339 bp shorter than that from SGII; the length of the PCR fragment from SGIIV2 will be 259bp shorter than that from SGII; the length of the PCR fragment from SGIIV3 will be 533 bp shorter than that from SGII.

Preferably, the primers of the invention contain 20 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of gene variants (SGIIV 1, SGIIV2 and SGIIV3) can be used. PCR fragments are analyzed on a 1% agarose gel using five microliters (10%) of the amplified products. The intensity of the signals may be determined by using the Molecular Analyst program (version 1.4.1; Bio-Rad). Thus, the index of relative expression levels for each co-amplified PCR products may be calculated based on the intensity of signals.

The RT-PCR experiment may be performed according to the manufacturer's instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5X RT-PCR buffer, 1μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of human SGII genes is performed by detecting the nucleotide sequence of SGIIV1, SGIIV2 or SGIIV3, which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid selected from the group consisting of SEQ ID NOs: 1, 3 and 5, and the fragments thereof; and (4) detecting whether the cDNA sample hybridizes with the nucleic acid of SEQ ID NOs: 1, 3 or 5, or the fragments thereof. If necessary, the amount of hybridized sample may be detected.

The expression of gene variants can be analyzed using the Northern Blot hybridization approach. Specific fragments comprising nucleotide 253-258, preferably nucleotides 240-269 of the SGIIV1, nucleotides 273-278, preferably nucleotides 261-290 of the SGIIV2 or nucleotides 1424-1429, preferably nucleotides 1413-1442 of the SGIIV3 may be amplified by polymerase chain reaction (PCR) using a primer set designed for RT-PCR. The amplified PCR fragment may be labeled and serve as a probe to hybridize the membranes containing the total RNAs extracted from the samples under the conditions of 55° C. in a suitable hybridization solution for 3 hours. Blots may be washed twice in 2×SSC, 0.1% SDS at room temperature for 15 minutes each, followed by two washes in 0.1×SSC and 0.1% SDS at 65° C. for 20 minutes each. After these washes, the blots may be rinsed briefly in a suitable washing buffer and incubated in a blocking solution for 30 minutes, and then incubated in a suitable antibody solution for 30 minutes. The blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be performed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency of human SGII gene may also be performed by detecting the polypeptides encoded by SGIIV1, SGIIV2 and SGIIV3 of the invention. For instance, the polypeptides in protein samples obtained from the mammal may be determined by, but is not limited to, the immunoassay, wherein the antibody specifically binding to the polypeptides of the invention is contacted with the protein sample, and the antibody-polypeptide complex is detected. If necessary, the amount of the antibody-polypeptide complexes can be determined.

The polypeptides encoded by the gene variants may be expressed in prokaryotic cells by using suitable prokaryotic expression vectors. The cDNA fragment of SGIIV1, SGIIV2 or SGIIV3 gene encoding the amino acid coding sequence may be PCR amplified with restriction enzyme digestion sites incorporated in the 5′ and 3′ ends, respectively. For example, the fragments comprising nucleotides 240-269 (encoding amino acid residues 60-69) of the SGIIV1, nucleotides 261-290 (encoding amino acid residues 67-76) or nucleotides 276-356 (encoding amino acid residues 72-98) of the SGIIV2 or nucleotides 1413-1442 (encoding amino acid residues 451-460) or nucleotides 1425-1478 (encoding amino acid residues 455-472) of the SGIIV3 may be PCR amplified. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 hours. The bacterially-expressed proteins may be purified.

The polypeptides encoded by SGII-related gene variants may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectamine Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins/polypeptides. After these proteins/polypeptides are purified, monoclonal antibodies against these purified proteins/polypeptides (SGIIV1, SGIIV2 and SGIIV3) may be generated using hybridoma technique according to the conventional methods (de StGroth and Scheidegger, (1980) J Immunol Methods 35:1-21; Cote et al. (1983) Proc Natl Acad Sci U S A 80: 2026-30; and Kozbor et al. (1985) J Immunol Methods 81:31-42).

According to the present invention, the presence of the polypeptides encoded by the gene variants in samples of lung cancers may be determined by, but is not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 hour at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with monoclonal antibody directed against the polypeptides encoded by the gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human clones from a SCLC cDNA library were compiled and served as an EST database. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative SGII encoding gene were identified during the course of EST generation.

### Isolation of cDNA Clones

Three cDNA clones exhibiting EST sequences similar to the SGII gene were isolated from the cDNA library and named SGIIV1, SGIIV2 and SGIIV3. The inserts of these clones were subsequently excised in vivo from the λZAP Express vector using the ExAssist/XLOLR helper phage system (Stratagene). Phagemid particles were excised by coinfecting XL1-BLUE MRF’ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### References

Altschul et al., Gapped BLAST and PSI-BLAST: a new generation of protein database search programs, Nucleic Acids Res, 25: 3389-3402, (1997).
Ausubel et al., Current protocols in Molecular Biology, John Wiley & Sons, New York N.Y., ch. 9, 13, and 16, (1995).
Boguski et al., dbEST—database for “expressed sequence tags”. Nat Genet. 4: 332-3, (1993).
Carney, D. N. The biology of lung cancer. Curr. Opin. Oncol. 4: 292-8, (1992a).
Carney, D. N. Biology of small-cell lung cancer. Lancet 339: 843-6, (1992b).
Cote et al., Generation of human monoclonal antibodies reactive with cellular antigens, Proc Natl Acad Sci U S A 80: 2026-30, (1983).
de StGroth and Scheidegger, Production of monoclonal antibodies: strategy and tactics, J Immunol Methods 35:1-21, (1980).
Gerdes et al., The primary structure of human secretogranin II, a widespread tyrosine-sulfated secretory granule protein that exhibits low pH- and calcium-induced aggregation. J Biol Chem 264:12009-15, (1989).
Gerdes et al., Nucleotide Accession No. M25756
Gish and States, Identification of protein coding regions by database similarity search, Nat Genet, 3:266-272, (1993).
Ihde and Minna, Non-small cell lung cancer. Part II: Treatment. Curr. Probl. Cancer 15: 105-54, (1991).
Kozbor et al., Specific immunoglobulin production and enhanced tumorigenicity following ascites growth of human hybridomas, J Immunol Methods, 81:31-42 (1985).
Liu et al., Silent mutation induces exon skipping of fibrillin-1 gene in Marfan syndrome. Nat Genet 16:328-9, (1997).
Lukas et al., Alternative and aberrant messenger RNA splicing of the mdm2 oncogene in invasive breast cancer. Cancer Res 61:3212-9, (2001).
Roberge et al., A strategy for a convergent synthesis of N-linked glycopeptides on a solid support. Science 269:202-4, (1995).
Sambrook, J. Cold Spring Harbor Press, Plainview N.Y., ch. 4, 8, and 16-17.
Sethi, Science, medicine, and the future. Lung cancer, BMJ, 314: 652-655, (1997)
Siffert et al., Association of a human G-protein beta3 subunit variant with hypertension. Nat Genet, 18:45-8, (1998).
Smyth et al., The impact of chemotherapy on small cell carcinoma of the bronchus. Q J Med, 61: 969-76, (1986).
Stallings-Mann et al., Alternative splicing of exon 3 of the human growth hormone receptor is the result of an unusual genetic polymorphism. Proc Natl Acad Sci U S A 93:12394-9, (1996).
Strausberg, R. EST Accession No. A1655028, A1671205, AA936920
Taupenot et al. The chromogranin-secretogranin family. N Engl J Med. 348:1134-49, (2003).

1. An isolated polypeptide comprising an amino acid sequence selected from the group consisting of SEQ ID NOs; 2, 4, and 6, and fragments thereof.

2. The isolated polypeptide of claim 1, wherein the fragments comprise the amino acid residues 60 to 69 of SEQ ID NO.: 2.

3. The isolated polypeptide of claim 1, wherein the fragments comprise the amino acid residues 67 to 76 or 72 to 98 of SEQ ID NO: 4.

4. The isolated polypeptide of claim 1, wherein the fragments comprise the amino acid residues 451 to 460 or 455 to 472 of SEQ ID NO: 6.

5. An isolated nucleic acid comprising a nucleotide sequence selected from the group consisting of SE ID NOs: 1, 3, and 5, and fragments thereof.

6. The isolated nucleic acid of claim 5, wherein the fragments comprise nucleotides 253 to 258 of SEQ ID NO: 1.

7. The isolated nucleic acid of claim 5, wherein the fragments comprise nucleotides 273 to 278 of SEQ ID NO: 3.

8. The isolated nucleic acid of claim 5, wherein the fragments comprise nucleotides 1424 to 1429 of SEQ ID NO: 5.

9. An expression vector comprising the nucleic acid of claim 5.

10. A host cell transformed with the expression vector of claim 9.

11. A method of producing a polypeptide, which comprises the steps of:
(1) culturing the host cell of claim 10 under a condition suitable for the expression of the polypeptide; and
(2) recovering the polypeptide from the host cell culture.

12. An antibody specifically binding to the polypeptide of claim 1.

13. A method for diagnosing a disease associated with the deficiency of a SGII gene in a mammal, which comprises detecting the nucleic acid of claim 5 or a polypeptide encoded thereby.

14. The method of claim 13, wherein the detection of the nucleic acid comprises the steps of:
(1) extracting total RNA from a sample obtained from the mammal;
(2) amplifying the RNA by reverse transcriptase-polymerase chain reaction (RT-PCR) to obtain a cDNA sample;
(3) bringing the cDNA sample into contact with the nucleic acid; and
(4) detecting whether the cDNA sample hybridizes with the nucleic acid.

15. The method of claim 14, further comprising the step of determining the amount of the hybridized sample.

16. The method of claim 13, wherein the detection of the nucleic acid comprises the steps of:
(1) extracting the total RNAs of cells obtained from the mammal;
(2) amplifying the RNA by reverse transcriptase-polymerase chain reaction (RT-PCR) with a set of primers to obtain a cDNA comprising the fragments comprising nucleotides 253 to 258 of SEQ ID NO: 1 or nucleotides 273 to 278 of SEQ ID NO: 3 or nucleotides 1424 to 1429 of SEQ ID NO: 5; and
(3) detecting whether the cDNA is obtained.

17. The method of claim 13, wherein the detection of the nucleic acid comprises the steps of:
(1) extracting the total RNAs of cells obtained from the mammal;
(2) amplifying the RNA by reverse transcriptase-polymerase chain reaction (RT-PCR) with a set of primers to obtain a cDNA comprising the fragments comprising nucleotides 240 to 269 of SEQ ID NO: 1 or nucleotides 261 to 290 of SEQ ID NO: 3 or nucleotides 1413 to 1442 of SEQ ID NO: 5; and
(3) detecting whether the cDNA is obtained.

18. The method of claim 16, wherein the forward primer has a sequence comprising the nucleotides 253 to 258 of SEQ ID NO: 1 and the reverse primer has a sequence complementary to the nucleotides of SEQ ID NO: 1 at any other locations downstream of nucleotide 258, or alternatively, the reverse primer has a sequence complementary to the nucleotides of SEQ ID NO: 1 containing nucleotides 253 to 258 and the forward primer has a sequence comprising the nucleotides of SEQ ID NO: 1 at any other locations upstream of nucleotide 253.

19. The method of claim 17, wherein the forward primer has a sequence comprising the nucleotides 240 to 269 of SEQ ID NO: 1 and the reverse primer has a sequence complementary to the nucleotides of SEQ ID NO: 1 at any other locations downstream of nucleotide 269, or alternatively, the reverse primer has a sequence complementary to the nucleotides of SEQ ID NO: 1 containing nucleotides 240 to 269 and the forward primer has a sequence comprising the nucleotides of SEQ ID NO: 1 at any other locations upstream of nucleotide 240.

20. The method of claim 16, wherein the forward primer has a sequence comprising the nucleotides 273 to 278 of SEQ ID NO: 3 and the reverse primer has a sequence complementary to the sequence complementary to the nucleotides of SEQ ID NO: 3 at any other locations downstream of nucleotide 278, or alternatively, the reverse primer has a sequence complementary to the nucleotides of SEQ ID NO: 3 containing nucleotides 273 to 278 and the forward primer has a sequence comprising the nucleotides of SEQ ID NO: 3 at any other locations upstream of nucleotide 273.

21. The method of claim 17, wherein the forward primer has a sequence comprising the nucleotides 261 to 290 of SEQ ID NO: 3 and the reverse primer has a sequence complementary to the sequence complementary to the nucleotides of SEQ ID NO: 3 at any other locations downstream of nucleotide 290, or alternatively, the reverse primer has a sequence complementary to the nucleotides of SEQ ID NO: 3 containing nucleotides 261 to 290 and the forward primer has a sequence comprising the nucleotides of SEQ ID NO: 3 at any other locations upstream of nucleotide 261.

22. The method of claim 16, wherein the forward primer has a sequence comprising the nucleotides 1424 to 1429 of SEQ ID NO: 5 and the reverse primer has a sequence complementary to the nucleotides of SEQ ID NO: 5 at any other locations downstream of nucleotide 1429, or alternatively, the reverse primer has a sequence complementary to the nucleotides of SEQ ID NO: 5 containing nucleotides 1424 to 1429 and the forward primer has a sequence comprising the nucleotides of SEQ ID NO: 5 at any other locations upstream of nucleotide 1424.

23. The method of claim 17, wherein the forward primer has a sequence comprising the nucleotides 1413 to 1442 of SEQ ID NO: 5 and the reverse primer has a sequence complementary to the nucleotides of SEQ ID NO: 5 at any other locations downstream of nucleotide 1442, or alternatively, the reverse primer has a sequence complementary to the nucleotides of SEQ ID NO: 5 containing nucleotides 1413 to 1442 and the forward primer has a sequence comprising the nucleotides of SEQ ID NO: 5 at any other locations upstream of nucleotide 1413.

24. The method of claim 16, wherein the forward primer has a sequence comprising the nucleotides of SEQ ID NO: 1 at any other locations upstream of nucleotide 253 and the reverse primer has a sequence complementary to the nucleotides of SEQ ID NO: 1 at any other locations downstream of nucleotide 258.

25. The method of claim 16, wherein the forward primer has a sequence comprising the nucleotides of SEQ ID NO: 3 at any other locations upstream of nucleotide 273 and the reverse primer has a sequence complementary to the nucleotides of SEQ ID NO: 3 at any other locations downstream of nucleotide 278.

26. The method of claim 16, wherein the forward primer has a sequence the nucleotides of SEQ ID NO: 5 at any other locations upstream of nucleotide 1424 and the reverse primer has a sequence complementary to the nucleotides of SEQ ID NO: 5 at any other locations downstream of nucleotide 1429.

27. The method of claim 24, the cDNA sample amplified from SEQ ID NO: 1 is 339 bp shorter than that from SGII.

28. The method of claim 25, the cDNA sample amplified from SEQ ID NO: 3 is 259 bp shorter than that from SGII.

29. The method of claim 26, the cDNA sample amplified from SEQ ID NO: 5 is 533 bp shorter than that from SGII.

30. The method of claim 16, further comprising the step of detecting the amount of the amplified cDNA sample.

31. The method of claim 13, wherein the detection of the polypeptide comprises the steps of contacting a antibody that specifically binds to the polypeptide, with protein samples extracted from the mammal, and detecting whether an antibody-polypeptide complex is formed.

32. The method of claim 31, further comprising the step of determining the amount of the antibody-polypeptide complex.
